SEQUENCE LISTING (12 sequences)

SEQ ID NO: 1 - DNA, 5314 bp, Plasmodium falciparum
aaaaaaacaaagaaacaaaaaagagatgtgaataagtttatgatgtgaataaatttatgatgtgtataaatttatgatgtgaataaatttatgatgtgaataaatttatgatgtgaataaatttatgatgtgtataaatttatgatgtgaataaatttatgatgtgaataaatttatgatgtgaataaatttatgatgtgaataaatttatgatgtgtataaatttatgatgtgtataaatttatgatgtgtataaatttatgatgtgtatatatttatgatgtgaataaatttatgatgtgtataaatttatgatgtgaataaatttatgatgtgtataaatttatgatgtgtatttatttttcttttttttagttttatataattatacatacatatatatatatatatatatttatatatatatacaaagggatgtttctaatatggcgtttgtataaaaccttattcttaatattgtatattataattttacaagaatatgtatgtcattcttctaatcatataaaaagtgttaatagatgttttatcccctttatttcagaatatatatataaacagaaaaacagaaaaaataatatatcgtatgattctagtaacagtcactacaatgataaaataataccatgtggtaattataataaacaattaaaatataaaataaatttttgtcgtaatttgtattttaataataaaaatgaatataatagaatatgctcaaggaacaaattaaattttcataatatacaaacagataatacaatatacaaaccaaaaaagaaatattatgaagttggaaaagtaagagaaaaaataaaaatgtataccctttttaaagatgacaaaataaacacactgaaatgtaatgaagaagaatctgttacttcagaaacaataaataagcatgattcaagtgtaaaagtaaaaggaagaagaaaaaaaaatataacaagtaatgaaaatattatgaatccaaataataaaagtgtaagtagcattaatacaaatctaagtgattcttctaataataaaaatgataattccttaaatagtaaaaaaaatgataataccttaaatagtaaaaaaaatgataataccttaaatagtaaaaaaaatgataataccttaaatagtaaaaataataacaattccttaaatagtaaaaataataacaattccttaaatagtaaaaagagtaataattcttataatgacaaacatatagaccatataattcctgaaggcaaaaataaaataaacaataatatagatgtaaaacacaatattaataacaaattaaatgaaattaatgaagaaccttatgaagatacacataataaagaagagaattcttcaaataaaaatgataatgatgaaaaaagaaaagaagaaaataataatataacaagaagatatataaaaaatgaccaaatgtcatataataatataaatacaaattctaatgaatatgacaaaaatgcatctacattagatgaaacatatataggtaaaacttttgaaggttatgtttatagtgttaataaaaatgctgcgtgtattaaattaaaaaatattaataaatatggtttgttatttaaaaacaaagcaaatttaggtgatgacattgaagatatgaatgatttttttgaaaaagatcaaccggttcatgtgaaaatacttggtattaatacaaagaagaatatattttatctaggaaatattataaaatataatgaaaatataaaattaagtaaaggagaatattcaaagggattaataacaaaagtatgtgattcctactgttttattaaagttttaaaaaatggaagcactggatatttacataaatcaaaattgttttgtatgaatgataaagaaaagaaaaataatgataatcaaaattatgataatccaaataatgataatccaaattatgataatcaaaattatgataatcaaaattataataatccaaattatgatcatccaaattatgataatcaaaattatgataatcaaaattataataatccaaataatgattatagtacatcacaattatataacagtgataatttacaaatggattttatatataaattacaatttacaaaaatatttaatatatgggatataatagatgttgaaatattaggaactccacaaaatgattataaatcaaattatatattgacaataccaagaggatctaaaacatttaagaaaattttgaattatttgaatgttttaaaagaaaatgaagatattaataatatacaatataaaggtgattatatctattctattgataataataaaaatgacaatataatagattcagatataaataatcatcacattaataataagaagaaaaagaagaatctatatgatatacaaaataatatgaatcattctccttttaataagtttcatacagaagatgaatatttatttaatgaccatgtccaagaaaatgttcacaccttttatgaaaaaaataaaaaatataaaattacatatgataaagaaaataatcataaaatgaataaatcgtattatttaaaaaaaaataaagaattaccttttaataataaattcaaaaaaatcattaaaaatatttatgaccttcctaacactatttctttatctatgttatctaaaacaattaaaataccattggcatctataaaaaaatattttatcatccacgaaaataaagaatataattcaagttataaaattaattcagaacagataaaacgaatatgtcaacatttcaaaatagactgtaatgtagaacagagagatgataatgtggttacaaaagtgaatgggacaactaaggattgtcaagaaaaagtttttaaaaatgtgacacaggacagattgaaggaaggtgaacaggaacgagttattaaggtggaagcaaagattaaaaatgatgaaatggttatgcaagagcaaaaagatactaaggaggagaagcacatggacgttcaatttattgaagaaaaagatattaatgtacaacatattaatgtacaagatatggatgtacaagatatggatgtacaagatattaatgtacaagatatggatgtgcaaaatattaatgtacaagatattaatattcaagatatggatgtgcaaaatattaataacagtataacacttaacaaatcgacaagttgtcaaaccgatgaatcgcgagacgcaccggggggtgaccaaaatgaatcgctcgatgaaaaagattcgatggaaaaaagtaaagaaaaaaaaaaaaaaaaagggaaaagtagaaaaaaaaataaagataccaatttaacattaaaaagtgatagtattcaaaaatcaaagaccaccctagacgataaaaaacgaaatgtggtagttacatttataggacatattaatcatggaaaaacgtctttatttgattatatatgtaaaaccaatgaacaaaaaaaggagtatggacttataacacaaaatataagagcgtttaaagcaaccgtaaggaataattttacatttactcttgtcgataccccaggacatgaagcatttatgcctatgagaagtagaggtgttaaaatatcagatttaagtattcttgttatatcaggagatgaaggaatacaagaacagactgtggaatgtataaaattaataaaagaatttaatattaaaattattattgcaataactaaagtagatattcctaatgttgatgtagatagaataattaatgatttgttatatcatgatataacaacagaattaaatggaggggaaatacaagtagttgaatgttctatttataaagaagaaagtatagataaattattagatgccatatatttagaatctgaatttcttaatctacaaaccaatcctgataagaaacatgaacaggctcaaggtgttgttttagattcttatatagataaaaatggaattgtttctataaatttgttacaaaatggtgtattaaatataaatgatcatttttatactgggtcatcatatggaaaagtgaagatattaaaagatcatttaaataaaaatattaaaagtgcatatccatcggatcctattaaaattattggatacaacaaaaattctgttcctgtagcaggtgacaaattttatgttgttgaaaatgaagccctagccaaagaaattgcggaacataataagaataaaatgttaacaatggaaattaataattttacttatgatcagacaaatatgaacaggtataaagattttataatatccagagaaaataaaattggaggttcttcaggtatactgggagaaaataatttaaaaaatgacattgatggtaatatgacaagggatgataatatgacaagggatgataatatgacaagtgatgataatatgacaagggatggtaatagaacaaataatgacaatattacaagtgatgataatatgtcaaatgattatgataaaataaaagaaacgaaaatgtatacaaataataaatcatttcaaaaggatgattttttaaaaatacatttgaataatacaaacgaaaatgtgattaatatggatccttcaacacatataggaaaaaatgaaataaaaacaatatactcaaattatattattaaatgtgataaacaaggaagtatagaagttttgaaaaattgtatgcttaaattacaaaaagaagatagtatatgtaaaataaaaaacaaaattatatatgctgatataggtaatgtaacatcaagtgatataaaatatgctacaagttttaatgctacaataatagcttttggtgttaaattatcaaatgatattaaaggttcaaaaaattcaaaaggttcaaaaaatcataataattatcctattatatattcaaatgtcttatatgaacttatagaaaatgtggaaaaagaaatggaaaagaaattaagtaaaaaaccaatgggtgaattaaaaggaacagcacaaattttaaaagtttttaatatatcgaaacttggaaaggttgcagggtgtattgttaaaaaaggtactatctctataaatagtaatattcgtattttaagaaatgataaagttatttatatgggaaaaatcatttctattaaaattgttaaggaagaaaaaacacaagtaacggaagctgatgaatgtggtataggttttgataattttttggattttgagccaaacgatataatcgaagcgtacgaaaattaaatatgaaaaaaaaaaaaaaaaatgtaatgtaaaaaaaaaaaaaaaa

SEQ ID NO: 2 - DNA, 1910 bp, Plasmodium falciparum
aaaaaaacaaagaaacaaaaaagagatgtgaataagtttatgatgtgaataaatttatgatgtgtataaatttatgatgtgaataaatttatgatgtgaataaatttatgatgtgaataaatttatgatgtgtataaatttatgatgtgaataaatttatgatgtgaataaatttatgatgtgaataaatttatgatgtgaataaatttatgatgtgtataaatttatgatgtgtataaatttatgatgtgtataaatttatgatgtgtatatatttatgatgtgaataaatttatgatgtgtataaatttatgatgtgaataaatttatgatgtgtataaatttatgatgtgtatttatttttcttttttttagttttatataattatacatacatatatatatatatatatatttatatatatatacaaagggatgtttctaatatggcgtttgtataaaaccttattcttaatattgtatattataattttacaagaatatgtatgtcattcttctaatcatataaaaagtgttaatagatgttttatcccctttatttcagaatatatatataaacagaaaaacagaaaaaataatatatcgtatgattctagtaacagtcactacaatgataaaataataccatgtggtaattataataaacaattaaaatataaaataaatttttgtcgtaatttgtattttaataataaaaatgaatataatagaatatgctcaaggaacaaattaaattttcataatatacaaacagataatacaatatacaaaccaaaaaagaaatattatgaagttggaaaagtaagagaaaaaataaaaatgtataccctttttaaagatgacaaaataaacacactgaaatgtaatgaagaagaatctgttacttcagaaacaataaataagcatgattcaagtgtaaaagtaaaaggaagaagaaaaaaaaatataacaagtaatgaaaatattatgaatccaaataataaaagtgtaagtagcattaatacaaatctaagtgattcttctaataataaaaatgataattccttaaatagtaaaaaaaatgataataccttaaatagtaaaaaaaatgataataccttaaatagtaaaaaaaatgataataccttaaatagtaaaaataataacaattccttaaatagtaaaaataataacaattccttaaatagtaaaaagagtaataattcttataatgacaaacatatagaccatataattcctgaaggcaaaaataaaataaacaataatatagatgtaaaacacaatattaataacaaattaaatgaaattaatgaagaaccttatgaagatacacataataaagaagagaattcttcaaataaaaatgataatgatgaaaaaagaaaagaagaaaataataatataacaagaagatatataaaaaatgaccaaatgtcatataataatataaatacaaattctaatgaatatgacaaaaatgcatctacattagatgaaacatatataggtaaaacttttgaaggttatgtttatagtgttaataaaaatgctgcgtgtattaaattaaaaaatattaataaatatggtttgttatttaaaaacaaagcaaatttaggtgatgacattgaagatatgaatgatttttttgaaaaagatcaaccggttcatgtgaaaatacttggtattaatacaaagaagaatatattttatctaggaaatattataaaatataatgaaaatataaaattaagtaaaggagaatattcaaagggattaataacaaaagtatgtgattcctactgttttattaaagttttaaaaaatggaagcactggatatttacataaatcaaaattgttttgtatgaatgataaagaaaagaaaaataa

SEQ ID NO: 3 - DNA, 2017 bp, Plasmodium falciparum
acacaatattaataacaaattaaatgaaattaatgaagaaccttatgaagatacacataataaagaagagaattcttcaaataaaaatgataatgatgaaaaaagaaaagaagaaaataataatataacaagaagatatataaaaaatgaccaaatgtcatataataatataaatacaaattctaatgaatatgacaaaaatgcatctacattagatgaaacatatataggtaaaacttttgaaggttatgtttatagtgttaataaaaatgctgcgtgtattaaattaaaaaatattaataaatatggtttgttatttaaaaacaaagcaaatttaggtgatgacattgaagatatgaatgatttttttgaaaaagatcaaccggttcatgtgaaaatacttggtattaatacaaagaagaatatattttatctaggaaatattataaaatataatgaaaatataaaattaagtaaaggagaatattcaaagggattaataacaaaagtatgtgattcctactgttttattaaagttttaaaaaatggaagcactggatatttacataaatcaaaattgttttgtatgaatgataaagaaaagaaaaataatgataatcaaaattatgataatccaaataatgataatccaaattatgataatcaaaattatgataatcaaaattataataatccaaattatgatcatccaaattatgataatcaaaattatgataatcaaaattataataatccaaataatgattatagtacatcacaattatataacagtgataatttacaaatggattttatatataaattacaatttacaaaaatatttaatatatgggatataatagatgttgaaatattaggaactccacaaaatgattataaatcaaattatatattgacaataccaagaggatctaaaacatttaagaaaattttgaattatttgaatgttttaaaagaaaatgaagatattaataatatacaatataaaggtgattatatctattctattgataataataaaaatgacaatataatagattcagatataaataatcatcacattaataataagaagaaaaagaagaatctatatgatatacaaaataatatgaatcattctccttttaataagtttcatacagaagatgaatatttatttaatgaccatgtccaagaaaatgttcacaccttttatgaaaaaaataaaaaatataaaattacatatgataaagaaaataatcataaaatgaataaatcgtattatttaaaaaaaaataaagaattaccttttaataataaattcaaaaaaatcattaaaaatatttatgaccttcctaacactatttctttatctatgttatctaaaacaattaaaataccattggcatctataaaaaaatattttatcatccacgaaaataaagaatataattcaagttataaaattaattcagaacagataaaacgaatatgtcaacatttcaaaatagactgtaatgtagaacagagagatgataatgtggttacaaaagtgaatgggacaactaaggattgtcaagaaaaagtttttaaaaatgtgacacaggacagattgaaggaaggtgaacaggaacgagttattaaggtggaagcaaagattaaaaatgatgaaatggttatgcaagagcaaaaagatactaaggaggagaagcacatggacgttcaatttattgaagaaaaagatattaatgtacaacatattaatgtacaagatatggatgtacaagatatggatgtacaagatattaatgtacaagatatggatgtgcaaaatattaatgtacaagatattaatattcaagatatggatgtgcaaaatattaataacagtataacacttaacaaatcgacaagttgtcaaaccgatgaatcgcgagacgcaccggggggtgaccaaaatgaatcgctcgatgaaaaagattcgatggaaaaaagtaaagaaaaaaaaaaaaaaaaa

SEQ ID NO: 4 - DNA, 2828 bp, Plasmodium falciparum
tccaagaaaatgttcacaccttttatgaaaaaaataaaaaatataaaattacatatgataaagaaaataatcataaaatgaataaatcgtattatttaaaaaaaaataaagaattaccttttaataataaattcaaaaaaatcattaaaaatatttatgaccttcctaacactatttctttatctatgttatctaaaacaattaaaataccattggcatctataaaaaaatattttatcatccacgaaaataaagaatataattcaagttataaaattaattcagaacagataaaacgaatatgtcaacatttcaaaatagactgtaatgtagaacagagagatgataatgtggttacaaaagtgaatgggacaactaaggattgtcaagaaaaagtttttaaaaatgtgacacaggacagattgaaggaaggtgaacaggaacgagttattaaggtggaagcaaagattaaaaatgatgaaatggttatgcaagagcaaaaagatactaaggaggagaagcacatggacgttcaatttattgaagaaaaagatattaatgtacaacatattaatgtacaagatatggatgtacaagatatggatgtacaagatattaatgtacaagatatggatgtgcaaaatattaatgtacaagatattaatattcaagatatggatgtgcaaaatattaataacagtataacacttaacaaatcgacaagttgtcaaaccgatgaatcgcgagacgcaccggggggtgaccaaaatgaatcgctcgatgaaaaagattcgatggaaaaaagtaaagaaaaaaaaaaaaaaaaagggaaaagtagaaaaaaaaataaagataccaatttaacattaaaaagtgatagtattcaaaaatcaaagaccaccctagacgataaaaaacgaaatgtggtagttacatttataggacatattaatcatggaaaaacgtctttatttgattatatatgtaaaaccaatgaacaaaaaaaggagtatggacttataacacaaaatataagagcgtttaaagcaaccgtaaggaataattttacatttactcttgtcgataccccaggacatgaagcatttatgcctatgagaagtagaggtgttaaaatatcagatttaagtattcttgttatatcaggagatgaaggaatacaagaacagactgtggaatgtataaaattaataaaagaatttaatattaaaattattattgcaataactaaagtagatattcctaatgttgatgtagatagaataattaatgatttgttatatcatgatataacaacagaattaaatggaggggaaatacaagtagttgaatgttctatttataaagaagaaagtatagataaattattagatgccatatatttagaatctgaatttcttaatctacaaaccaatcctgataagaaacatgaacaggctcaaggtgttgttttagattcttatatagataaaaatggaattgtttctataaatttgttacaaaatggtgtattaaatataaatgatcatttttatactgggtcatcatatggaaaagtgaagatattaaaagatcatttaaataaaaatattaaaagtgcatatccatcggatcctattaaaattattggatacaacaaaaattctgttcctgtagcaggtgacaaattttatgttgttgaaaatgaagccctagccaaagaaattgcggaacataataagaataaaatgttaacaatggaaattaataattttacttatgatcagacaaatatgaacaggtataaagattttataatatccagagaaaataaaattggaggttcttcaggtatactgggagaaaataatttaaaaaatgacattgatggtaatatgacaagggatgataatatgacaagggatgataatatgacaagtgatgataatatgacaagggatggtaatagaacaaataatgacaatattacaagtgatgataatatgtcaaatgattatgataaaataaaagaaacgaaaatgtatacaaataataaatcatttcaaaaggatgattttttaaaaatacatttgaataatacaaacgaaaatgtgattaatatggatccttcaacacatataggaaaaaatgaaataaaaacaatatactcaaattatattattaaatgtgataaacaaggaagtatagaagttttgaaaaattgtatgcttaaattacaaaaagaagatagtatatgtaaaataaaaaacaaaattatatatgctgatataggtaatgtaacatcaagtgatataaaatatgctacaagttttaatgctacaataatagcttttggtgttaaattatcaaatgatattaaaggttcaaaaaattcaaaaggttcaaaaaatcataataattatcctattatatattcaaatgtcttatatgaacttatagaaaatgtggaaaaagaaatggaaaagaaattaagtaaaaaaccaatgggtgaattaaaaggaacagcacaaattttaaaagtttttaatatatcgaaacttggaaaggttgcagggtgtattgttaaaaaaggtactatctctataaatagtaatattcgtattttaagaaatgataaagttatttatatgggaaaaatcatttctattaaaattgttaaggaagaaaaaacacaagtaacggaagctgatgaatgtggtataggttttgataattttttggattttgagccaaacgatataatcgaagcgtacgaaaattaaatatgaaaaaaaaaaaaaaaaatgtaatgtaaaaaaaaaaaaaaaaaa

SEQ ID NO: 5 - DNA, 496 bp, Plasmodium falciparum
gttacttcagaaacaataaataagcatgattcaagtgtaaaagtaaaaggaagaagaaaaaaaaatataacaagtaatgaaaatattatgaatccaaataataaaagtgtaagtagcattaatacaaatctaagtgattcttctaataataaaaatgataattccttaaatagtaaaaaaaatgataataccttaaatagtaaaaaaaatgataataccttaaatagtaaaaaaaatgataataccttaaatagtaaaaataataacaattccttaaatagtaaaaataataacaattccttaaatagtaaaaagagtaataattcttataatgacaaacatatagaccatataattcctgaaggcaaaaataaaataaacaataatatagatgtaaaacacaatattaataacaaattaaatgaaattaatgaagaaccttatgaagatacacataataaagaagagaattcttcaaataaaaatgataatgatg

SEQ ID NO: 6 - DNA, 1678 bp, Plasmodium falciparum
aaaaaaacaaagaaacaaaaaagagatgtgaataagtttatgatgtgaataaatttatgatgtgtataaatttatgatgtgaataaatttatgatgtgaataaatttatgatgtgaataaatttatgatgtgtataaatttatgatgtgaataaatttatgatgtgaataaatttatgatgtgaataaatttatgatgtgaataaatttatgatgtgtataaatttatgatgtgtataaatttatgatgtgtataaatttatgatgtgtatatatttatgatgtgaataaatttatgatgtgtataaatttatgatgtgaataaatttatgatgtgtataaatttatgatgtgtatttatttttcttttttttagttttatataattatacatacatatatatatatatatatatttatatatatatacaaagggatgtttctaatatggcgtttgtataaaaccttattcttaatattgtatattataattttacaagaatatgtatgtcattcttctaatcatataaaaagtgttaatagatgttttatcccctttatttcagaatatatatataaacagaaaaacagaaaaaataatatatcgtatgattctagtaacagtcactacaatgataaaataataccatgtggtaattataataaacaattaaaatataaaataaatttttgtcgtaatttgtattttaataataaaaatgaatataatagaatatgctcaaggaacaaattaaattttcataatatacaaacagataatacaatatacaaaccaaaaaagaaatattatgaagttggaaaagtaagagaaaaaataaaaatgtataccctttttaaagatgacaaaataaacacactgaaatgtaatgaagaagaatctgttacttcagaaacaataaataagcatgattcaagtgtaaaagtaaaaggaagaagaaaaaaaaatataacaagtaatgaaaatattatgaatccaaataataaaagtgtaagtagcattaatacaaatctaagtgattcttctaataataaaaatgataattccttaaatagtaaaaaaaatgataataccttaaatagtaaaaaaaatgataataccttaaatagtaaaaaaaatgataataccttaaatagtaaaaataataacaattccttaaatagtaaaaataataacaattccttaaatagtaaaaagagtaataattcttataatgacaaacatatagaccatataattcctgaaggcaaaaataaaataaacaataatatagatgtaaaacacaatattaataacaaattaaatgaaattaatgaagaaccttatgaagatacacataataaagaagagaattcttcaaataaaaatgataatgatgaaaaaagaaaagaagaaaataataatataacaagaagatatataaaaaatgaccaaatgtcatataataatataaatacaaattctaatgaatatgacaaaaatgcatctacattagatgaaacatatataggtaaaacttttgaaggttatgtttatagtgttaataaaaatgctgcgtgtattaaattaaaaaatattaataaatatggtttgttatttaaaaacaaagcaaatttaggtgatgacattgaagatatgaatgatttttttgaaaaagatc

SEQ ID NO: 7 - DNA, 1799 bp, Plasmodium falciparum
agatattaataatatacaatataaaggtgattatatctattctattgataataataaaaatgacaatataatagattcagatataaataatcatcacattaataataagaagaaaaagaagaatctatatgatatacaaaataatatgaatcattctccttttaataagtttcatacagaagatgaatatttatttaatgaccatgtccaagaaaatgttcacaccttttatgaaaaaaataaaaaatataaaattacatatgataaagaaaataatcataaaatgaataaatcgtattatttaaaaaaaaataaagaattaccttttaataataaattcaaaaaaatcattaaaaatatttatgaccttcctaacactatttctttatctatgttatctaaaacaattaaaataccattggcatctataaaaaaatattttatcatccacgaaaataaagaatataattcaagttataaaattaattcagaacagataaaacgaatatgtcaacatttcaaaatagactgtaatgtagaacagagagatgataatgtggttacaaaagtgaatgggacaactaaggattgtcaagaaaaagtttttaaaaatgtgacacaggacagattgaaggaaggtgaacaggaacgagttattaaggtggaagcaaagattaaaaatgatgaaatggttatgcaagagcaaaaagatactaaggaggagaagcacatggacgttcaatttattgaagaaaaagatattaatgtacaacatattaatgtacaagatatggatgtacaagatatggatgtacaagatattaatgtacaagatatggatgtgcaaaatattaatgtacaagatattaatattcaagatatggatgtgcaaaatattaataacagtataacacttaacaaatcgacaagttgtcaaaccgatgaatcgcgagacgcaccggggggtgaccaaaatgaatcgctcgatgaaaaagattcgatggaaaaaagtaaagaaaaaaaaaaaaaaaaagggaaaagtagaaaaaaaaataaagataccaatttaacattaaaaagtgatagtattcaaaaatcaaagaccaccctagacgataaaaaacgaaatgtggtagttacatttataggacatattaatcatggaaaaacgtctttatttgattatatatgtaaaaccaatgaacaaaaaaaggagtatggacttataacacaaaatataagagcgtttaaagcaaccgtaaggaataattttacatttactcttgtcgataccccaggacatgaagcatttatgcctatgagaagtagaggtgttaaaatatcagatttaagtattcttgttatatcaggagatgaaggaatacaagaacagactgtggaatgtataaaattaataaaagaatttaatattaaaattattattgcaataactaaagtagatattcctaatgttgatgtagatagaataattaatgatttgttatatcatgatataacaacagaattaaatggaggggaaatacaagtagttgaatgttctatttataaagaagaaagtatagataaattattagatgccatatatttagaatctgaatttcttaatctacaaaccaatcctgataagaaacatgaacaggctcaaggtgttgttttagattcttatatagataaaaatggaattgtttctataaatttgttacaaaatggtgtattaaatataaatgatcatttttatactgggtcatcatatggaaaagtgaagatattaaaagatc

SEQ ID NO: 8 - DNA, 693 bp, Plasmodium falciparum
ggatccttcaacacatataggaaaaaatgaaataaaaacaatatactcaaattatattattaaatgtgataaacaaggaagtatagaagttttgaaaaattgtatgcttaaattacaaaaagaagatagtatatgtaaaataaaaaacaaaattatatatgctgatataggtaatgtaacatcaagtgatataaaatatgctacaagttttaatgctacaataatagcttttggtgttaaattatcaaatgatattaaaggttcaaaaaattcaaaaggttcaaaaaatcataataattatcctattatatattcaaatgtcttatatgaacttatagaaaatgtggaaaaagaaatggaaaagaaattaagtaaaaaaccaatgggtgaattaaaaggaacagcacaaattttaaaagtttttaatatatcgaaacttggaaaggttgcagggtgtattgttaaaaaaggtactatctctataaatagtaatattcgtattttaagaaatgataaagttatttatatgggaaaaatcatttctattaaaattgttaaggaagaaaaaacacaagtaacggaagctgatgaatgtggtataggttttgataattttttggattttgagccaaacgatataatcgaagcgtacgaaaattaaatatgaaaaaaaaaaaaaaaaatgtaatgtaa

SEQ ID NO: 9 - protein, 1610 aa, Plasmodium falciparum
MFLIWRLYKTLFLILYIIILQEYVCHSSNHIKSVNRCFIPFISEYIYKQKNRKNNISYDSSNSHYNDKIIPCGNYNKQLKYKINFCRNLYFNNKNEYNRICSRNKLNFHNIQTDNTIYKPKKKYYEVGKVREKIKMYTLFKDDKINTLKCNEEESVTSETINKHDSSVKVKGRRKKNITSNENIMNPNNKSVSSINTNLSDSSNNKNDNSLNSKKNDNTLNSKKNDNTLNSKKNDNTLNSKNNNNSLNSKNNNNSLNSKKSNNSYNDKHIDHIIPEGKNKINNNIDVKHNINNKLNEINEEPYEDTHNKEENSSNKNDNDEKRKEENNNITRRYIKNDQMSYNNINTNSNEYDKNASTLDETYIGKTFEGYVYSVNKNAACIKLKNINKYGLLFKNKANLGDDIEDMNDFFEKDQPVHVKILGINTKKNIFYLGNIIKYNENIKLSKGEYSKGLITKVCDSYCFIKVLKNGSTGYLHKSKLFCMNDKEKKNNDNQNYDNPNNDNPNYDNQNYDNQNYNNPNYDHPNYDNQNYDNQNYNNPNNDYSTSQLYNSDNLQMDFIYKLQFTKIFNIWDIIDVEILGTPQNDYKSNYILTIPRGSKTFKKILNYLNVLKENEDINNIQYKGDYIYSIDNNKNDNIIDSDINNHHINNKKKKKNLYDIQNNMNHSPFNKFHTEDEYLFNDHVQENVHTFYEKNKKYKITYDKENNHKMNKSYYLKKNKELPFNNKFKKIIKNIYDLPNTISLSMLSKTIKIPLASIKKYFIIHENKEYNSSYKINSEQIKRICQHFKIDCNVEQRDDNVVTKVNGTTKDCQEKVFKNVTQDRLKEGEQERVIKVEAKIKNDEMVMQEQKDTKEEKHMDVQFIEEKDINVQHINVQDMDVQDMDVQDINVQDMDVQNINVQDINIQDMDVQNINNSITLNKSTSCQTDESRDAPGGDQNESLDEKDSMEKSKEKKKKKGKSRKKNKDTNLTLKSDSIQKSKTTLDDKKRNVVVTFIGHINHGKTSLFDYICKTNEQKKEYGLITQNIRAFKATVRNNFTFTLVDTPGHEAFMPMRSRGVKISDLSILVISGDEGIQEQTVECIKLIKEFNIKIIIAITKVDIPNVDVDRIINDLLYHDITTELNGGEIQVVECSIYKEESIDKLLDAIYLESEFLNLQTNPDKKHEQAQGVVLDSYIDKNGIVSINLLQNGVLNINDHFYTGSSYGKVKILKDHLNKNIKSAYPSDPIKIIGYNKNSVPVAGDKFYVVENEALAKEIAEHNKNKMLTMEINNFTYDQTNMNRYKDFIISRENKIGGSSGILGENNLKNDIDGNMTRDDNMTRDDNMTSDDNMTRDGNRTNNDNITSDDNMSNDYDKIKETKMYTNNKSFQKDDFLKIHLNNTNENVINMDPSTHIGKNEIKTIYSNYIIKCDKQGSIEVLKNCMLKLQKEDSICKIKNKIIYADIGNVTSSDIKYATSFNATIIAFGVKLSNDIKGSKNSKGSKNHNNYPIIYSNVLYELIENVEKEMEKKLSKKPMGELKGTAQILKVFNISKLGKVAGCIVKKGTISINSNIRILRNDKVIYMGKIISIKIVKEEKTQVTEADECGIGFDNFLDFEPNDIIEAYEN

SEQ ID NO: 10 - DNA, 951 bp, Plasmodium falciparum
atgtttctaatatggcgtttgtataaaaccttattcttaatattgtatattataattttacaagaatatgtatgtcattcttctaatcatataaaaagtgttaatagatgttttatcccctttatttcagaatatatatataaacagaaaaacagaaaaaataatatatcgtatgattctagtaacagtcactacaatgataaaataataccatgtggtaattataataaacaattaaaatataaaataaatttttgtcgtaatttgtattttaataataaaaatgaatataatagaatatgctcaaggaacaaattaaattttcataatatacaaacagataatacaatatacaaaccaaaaaagaaatattatgaagttggaaaagtaagagaaaaaataaaaatgtataccctttttaaagatgacaaaataaacacactgaaatgtaatgaagaagaatctgttacttcagaaacaataaataagcatgattcaagtgtaaaagtaaaaggaagaagaaaaaaaaatataacaagtaatgaaaatattatgaatccaaataataaaagtgtaagtagcattaatacaaatctaagtgattcttctaataataaaaatgataattccttaaatagtaaaaaaaatgataataccttaaatagtaaaaaaaatgataataccttaaatagtaaaaaaaatgataataccttaaatagtaaaaataataacaattccttaaatagtaaaaataataacaattccttaaatagtaaaaagagtaataattcttataatgacaaacatatagaccatataattcctgaaggcaaaaataaaataaacaataatatagatgtaaaacacaatattaataacaaattaaatgaaattaatgaagaaccttatgaagatacacataataaagaagagaattcttcaaataaaaaa

SEQ ID NO: 11 - DNA, 978 bp, Plasmodium falciparum
atgtttctaatatggcgtttgtataaaaccttattcttaatattgtatattataattttacaagaatatgtatgtcattcttctaatcatataaaaagtgttaatagatgttttatcccctttatttcagaatatatatataaacagaaaaacagaaaaaataatatatcgtatgattctagtaacagtcactacaatgataaaataataccatgtggtaattataataaacaattaaaatataaaataaatttttgtcgtaatttgtattttaataataaaaatgaatataatagaatatgctcaaggaacaaattaaattttcataatatacaaacagataatacaatatacaaaccaaaaaagaaatattatgaagttggaaaagtaagagaaaaaataaaaatgtataccctttttaaagatgacaaaataaacacactgaaatgtaatgaagaagaatctgttacttcagaaacaataaataagcatgattcaagtgtaaaagtaaaaggaagaagaaaaaaaaatataacaagtaatgaaaatattatgaatccaaataataaaagtgtaagtagcattaatacaaatctaagtgattcttctaataataaaaatgataattccttaaatagtaaaaaaaatgataataccttaaatagtaaaaaaaatgataataccttaaatagtaaaaaaaatgataataccttaaatagtaaaaaaaatgataataccttaaatagtaaaaataataacaattccttaaatagtaaaaataataacaattccttaaatagtaaaaagagtaataattcttataatgacaaacatatagaccatataattcctgaaggcaaaaataaaataaacaataatatagatgtaaaacacaatattaataacaaattaaatgaaattaatgaagaaccttatgaagatacacataataaagaagagaattcttcaaataaaaaa

SEQ ID NO: 12 - DNA, 978 bp, Plasmodium falciparum; identical to SEQ ID NO: 11

(19) United States
(12) Patent Application Publication
(10) Pub. No.: US 2003/0104003 A1
(43) Pub. Date: Jun. 5, 2003

(54) Novel surface protein of the malaria parasite plasmodium falciparum

(76) Inventors: Anthony A. James, Irvine, CA (US); Thanh V. Nguyen, San Diego, CA (US)

Correspondence Address: CHRISTIE, PARKER & HALE, LLP, P.O. Box 7068, Pasadena, CA, 91109-7068, US

(21) Appl. No.: 10/155,533

(22) Filed: May 24, 2002

Related U.S. Application Data
(60) Provisional application No. 60/293,633, filed on May 25, 2001.

(51) Int. Cl. A61K 39/002; C07H 21/04; C12N 9/00; C12N 1/10; C12N 15/74

(52) U.S. Cl. 424/191.1; 435/69.3; 435/320.1; 435/183; 435/258.2; 536/23.7

(57) ABSTRACT

Vaccines, antibodies, polypeptides, DNAs and RNAs for diagnosis, prophylaxis, treatment and detection of malaria or Plasmodium infection. P. falciparum antigen and fragments thereof and recombinant proteins or fusion proteins produced thereby. Methods for diagnosis, prophylaxis, treatment and detection of malaria or Plasmodium infection.

### CROSS-REFERENCE TO RELATED APPLICATION(S)

This application claims the benefit of U.S. Provisional Application Ser. No. 60/293,633, filed May 25, 2001, the content of which is hereby incorporated by reference in its entirety.

### FIELD OF THE INVENTION

This invention concerns vaccines, antibodies, proteins, DNAs and RNAs for diagnosis, prophylaxis and treatment of Plasmodium infections and for detection of Plasmodium. In particular, this invention concerns P. falciparum antigen comprised of a protein, as well as polyclonal and monoclonal antibodies directed against the antigen, DNA and RNA encoding the P. falciparum antigen and fragments and analogs thereof, and methods for production of recombinant or fusion proteins. This invention also concerns methods for diagnosis, prophylaxis, treatment of P. falciparum infections and detection of P. falciparum.

### BACKGROUND OF THE INVENTION

Plasmodium falciparum is the most virulent etiological agent of human malaria, responsible for over 90% of mortality due to the disease. Each year 300-500 million people are infected by malaria parasites, resulting in 1.5-3 million deaths (World Health Organization (1998) Fact Sheet N94, World Health Organization, Geneva, Switzerland). Efforts to eradicate malaria generally have failed, and currently the disease is endemic in more than 90 countries throughout the tropics. Widespread and increasing drug and insecticide resistance have exacerbated the situation, undermining the effectiveness of existing malaria control methods that depend on chemotherapy and vector control, respectively. Novel means to fight the disease are needed urgently, and a vaccine is predicted to have the greatest impact in addition to being the most cost-effective control measure (Miller, L. H., and Hoffman, S. L. (1998) Nat. Med. 4, 520-524).

Experimental support for the development of a vaccine for human malaria was provided first by the use of radiation-attenuated sporozoites as immunogens (Clyde, D. F., et al. (1973) Am. J. Med. Sci. 266, 169-177). The success of this experimental vaccination provided the impetus for the search for mechanisms of protective immune responses and the target antigens involved.

The circumsporozoite (CS) protein was identified as the major surface antigen of Plasmodium sporozoites (Yoshida, N., et al. (1980) Science 207, 71-73; Zavala, F., et al. (1983) J. Exp. Med. 157, 1947-1957; Dame, J. B., et al. (1984) Science 225, 593-599). The CS protein has been a leading vaccine candidate antigen because irradiated sporozoite-induced, protected human volunteers have high titers of anti-CS antibodies (Herrington, D., et al. (1991) Am. J. Trop. Med. Hyg. 5, 539-547), and CS-specific monoclonal antibodies and cytotoxic T-lymphocytes could adoptively transfer protection in a rodent malaria model system (Weiss, W. R., et al. (1992) J. Immunol. 149, 2103-2109). However, attempts to induce protection in humans using P. falciparum CS-based vaccines, despite recent improvement in their immunogenicity, have repeatedly yielded only partial success (Ballou, W. R., et al. (1987) Lancet 1, 1277-1281; Herrington, D., et al. (1987) Nature 328, 257-259; Hoffman, S. L., et al. (1993) in Molecular Immunologic Considerations in Malaria Vaccine Development, (Good, M. F., and Saul, A. J., eds) pp. 149-167, CRC Press, London; Stoute, J. A., et al. (1997) N. Engl. J. Med. 336, 86-91; Stoute, J. A., et al. (1998) J. Infect. Dis. 178, 1139-1144). The inability to develop a vaccine based on the CS protein has been interpreted to indicate that additional antigens play a role in irradiated sporozoite-mediated protection against infection (Galey, B., et al. (1990) Infect. Immun. 9, 2995-3001).

The SPf66 vaccine was the first multicomponent P. falciparum vaccine developed that contains peptide epitopes derived from a number of erythrocytic stage antigens (Patarroyo, M. E., et al. (1988) Nature 332, 158). These peptide epitopes were synthesized chemically and linked together by the repeat amino acid sequence of the pre-erythrocytic stage CS antigen. Although the SPf66 vaccine showed promising results in early trials, large-scale human trials revealed later that it provides only limited protection (Alonso, P. L., et al. (1994) Lancet 344, 1175; Alonso, P. L., et al. (1994) Vaccine 12, 181; D'Alessandro, U., et al. (1995) The Lancet 346, 462).

More recently, another multistage, multicomponent P. falciparum vaccine, NYVAC-Pf7, was developed (Tine, J. A., et al. (1996) Infection and Immunity 64, 3833). This vaccine formulation combined seven antigens that are known to be expressed in different developmental stages of the P. falciparum lifecycle. NYVAC-Pf7 was tested in human volunteers and disappointingly did not protect against malaria. Of the 35 volunteers challenged, only one did not develop malaria, although there was a significant delay in the onset of parasitemia in the remaining volunteers (Ockenhouse, C. F., et al. (1998) Journal of Infectious Disease 177, 1664). Interestingly, as with previous malaria vaccines, among all vaccinated individuals, protection or delay in the prepatent period did not correlate with antibody titers, CTL activity, or lymphoproliferative responses.

The failure of single-antigen CS-based and the multicomponent vaccines SPf66 and NYVAC-Pf7 may indicate that the vaccines lack essential epitopes required to induce the protective immunity that is as consistent and long-lasting as that observed in the irradiated-sporozoite vaccine.

Thus, there is a continuous need to identify antigens that may act independently, additively or synergistically with the CS protein in the development of a multicomponent vaccine.

Additionally, there is a need to have available methods for reproducible expression of specific target for P. falciparum antigen in large amounts, which antigen would provide a better immunogen. This approach requires that a specific P. falciparum antigen is cloned and identified as a potential candidate through its ability to elicit an antibody response that is immunoprotective. Before antibodies produced in this manner are tested in or administered to humans or animals, in vitro testing of their inhibitory effect on P. falciparum in cultured cells and in vivo studies would be desirable.

### SUMMARY OF THE INVENTION

One aspect of this invention concerns vaccines, antigens, antibodies, proteins, DNAs and RNAs for prophylaxis, treatment and detection or diagnosis of malaria or Plasmodium infections.

Another aspect of this invention concerns a P. falciparum antigen protein MB2 and fragments thereof.

Still another aspect of this invention concerns polyclonal or monoclonal antibodies directed against the P. falciparum antigen.

Still yet another aspect of this invention concerns DNA and RNA encoding the P. falciparum antigen and fragments thereof.

Still another aspect of this invention concerns a natural, synthetic or recombinant vaccine useful for active immunization of animals and humans against P. falciparum infection.

Still another aspect of this invention concerns a natural, synthetic or recombinant protein useful for preparation of passive immune products for treatment of established infection.

Another aspect of this invention concerns a natural, synthetic or recombinant DNA vaccine capable of endogenous production of inhibitory amount of anti-P. falciparum antibodies.

Another aspect of this invention concerns a natural, synthetic or recombinant RNA vaccine capable of endogenous development of inhibitory amount of anti-P. falciparum antibodies.

Still yet another aspect of the invention is the use of antigen, antibody, DNA or RNA to detect the presence of MB2 or antibodies to MB2, or DNA or RNA encoding MB2, for diagnosis in a human or animal host or detection in the environment.

Another aspect of this invention concerns the sequence of a 1610 amino acid protein of MW 120 kDa present in sporozoites and merozoites, and its amino acid and size variants.

Another aspect of this invention concerns the DNA sequence of 4830 nucleotides encoding the 120 kDa protein, its nucleotide and size variants and its upstream regulatory elements.

Another aspect of this invention concerns the RNA sequence determined by the DNA sequence of MB2 and its nucleotide and size variants including polyadenylation sequence.

Still yet another aspect of this invention concerns a group of MB2 recombinant or expressed protein targets of polyclonal antibodies which inhibit P. falciparum infection, invasion, or adhesion.

Another aspect of this invention concerns a method for prophylaxis and treatment of malaria or Plasmodium infections using vaccines, antibodies, proteins, DNAs and RNAs of the invention.

Still yet another aspect of this invention concerns a method of prophylaxis, treatment, inhibition or retardation of malaria or a Plasmodium infection comprising administering to a subject in need of such treatment an amount of an anti-P. falciparum polyclonal or monoclonal antibodies prophylactically or therapeutically effective to provide immunity against infection or treatment for disease.

Still yet another aspect of this invention concerns a method of prophylaxis, treatment, retardation, or inhibition of malaria or Plasmodium infection comprising administering to a subject in need of such treatment a vaccine comprising the polypeptide of this invention or its DNA or RNA capable of endogenous stimulation of the production of inhibitory amount of anti-P. falciparum antibodies or protective cellular immune responses.

Still yet another aspect of this invention concerns a method for diagnosing Plasmodium infection of a subject, comprising steps: (a) contacting a body specimen, fluid or tissue obtained from the subject with an anti-P. falciparum monoclonal or polyclonal antibody; and (b) detecting the formation of antibody-antigen complex wherein the presence of the complex indicates the presence of a P. falciparum organism in the subject.

Still yet another aspect of this invention concerns a method for detecting anti-P. falciparum antibody in a subject, said method comprising steps: (a) contacting a body specimen, fluid or tissue obtained from the subject with the MB2; and (b) detecting a formation of antibody-antigen complex wherein the presence of the complex indicates the presence of a P. falciparum antibody in the subject.

Still another aspect of this invention is a P. falciparum diagnostic or detection kit comprising anti-P. falciparum specific monoclonal and polyclonal antibodies or antigen according to the invention and a means for detection of an antibody-antigen complex.

Yet another aspect of the invention pertains to reagents resulting from activation of a cell mediated immune response to MB2 antigen, including cytokines and cytotoxic cells.

### BRIEF DESCRIPTION OF THE DRAWINGS

FIG. 1. Southern and Northern analyses of the MB2 gene and transcription product. A, Southern blot of P. falciparum genomic DNA, strain FCR3, digested with various restriction enzymes and hybridized with the spz-MB2 cDNA clone. Lane 1, EcoRI/HindIII; lane 2, PstI/HindIII; lane 3, PstI/EcoRI; lane 4, PstI/EcoRV; lane 5, PstI/NdeI. B, Northern blot of P. falciparum blood-stage mRNA hybridized with a probe derived from nucleotides 1-580 of the MB2 ORF. The lane contained 20 μg of total RNA. The approximate locations of molecular size markers in kilobases (kb) are indicated to the right of each of the panels.

FIG. 2. Structure of the MB2 gene and expression products. A, schematic representation of cDNA and genomic clones used to identify and assemble a cDNA containing the complete ORF of the MB2 gene. The cDNAs, spz-MB2, c3-1-18, c18-4-23, and c3-4-29, are represented as horizontal lines above a linear representation of the full-length MB2 cDNA. The numbers above each cDNA refer to the terminal nucleotide positions in the completed cDNA. The As in parentheses in the cDNA clones represent the internal and terminal priming poly(A) sites of the oligo(dT) primers. The full-length cDNA is represented as a horizontal line numbered with the positions of the translation initiation (ATG) and translation termination (TAA) codons, and the beginning and end of the sequence. The 5′ end untranslated region (5′-UTR) and polyadenylation sequences (A) also are indicated. The three horizontal lines at the bottom denote the MB2 genomic clones, g2-6-8, g2-4-4#5, and g6-2-2. The locations of the terminal nucleotides with respect to the cDNA are indicated above each line. Four horizontal arrows (a-d) represent the orientation and approximate location of gene amplification primers used to verify the contiguity of the sequence in the parasite genome. B, schematic representation of the MB2 protein sequence. The three domains, basic (B), acidic (A), and GTP-binding (G), are indicated as blocks with the junctions of the domains numbered below. The amino (H₂N) and carboxyl (COOH) ends are labeled. The four short horizontal lines represent the approximate extents of the polypeptides, MB2-B, MB2-C, MB2-FA, and MB2-IF2, used to generate antibodies. C, primary amino acid sequence of the conceptual translation of the MB2 gene. Amino acids in bold represent the putative signal peptide; bold and boxed, putative nuclear localization sequences; bold and italicized, repeat regions with a single repeat unit underlined; bold and underlined, cell-surface retention sequence; italicized and boxed, motifs conserved in the G domain.

FIG. 3. Immunolocalization of the MB2 protein in different developmental stages of P. falciparum. A-D: sporozoite preparations. A shows a cross-section of a sporozoite (S) in the mosquito salivary gland (Sg) reacted with anti-B domain antiserum. B is a cross-section of a free sporozoite reacted with anti-A domain antiserum. C and D are partial-oblique and cross sections (respectively) of sporozoites in HepG2-A16 cells (He) reacted with anti-B and anti-A domain antisera, respectively. PV is the parasitophorous vacuole space. E-G: asexual stage parasite preparations. E is a cross-section of a trophozoite in an erythrocyte (E), showing localization principally to the parasite nucleus (N) and some in the parasite cytoplasm (Pc). F is a section of schizonts showing MB2 localization to the nucleus and some cytoplasm. Hemazoin (Hz) also is visible. Both E and F were reacted with anti-B domain antiserum. G shows sections of parasites at the merozoite (Mz) stage reacted with anti-A domain antiserum, showing only cytoplasmic localization. H and I: localization of MB2 in gametocytes (labeled G) reacted with anti-B and anti-A domain antisera, respectively. MB2 can be detected in the nucleus, cytoplasm, and the PV space. Arrows indicate the location of gold particles. J: localization of MB2 in the exoerythrocytic (EE) stages of an Aotus monkey hepatocyte (AH) reacted with anti-B domain antiserum. All bars are 0.5 μm in length.

FIG. 4. Immunoblot analysis of protein extracts of P. falciparum sporozoite and blood stages. A and B: protein extracts prepared from sporozoites recovered from salivary glands of infected mosquitoes. C and D: proteins extracts prepared from asexual blood-stage parasites. A and C were probed with anti-B domain (MB2-B) antiserum; B and D were probed with anti-A domain (MB2-FA) antiserum. The molecular size markers (in kDa) are indicated to the left of each figure, and arrows to the right mark the locations of the MB2 polypeptides.

FIG. 5. Summary of expression and localization data for the MB2 protein. Each panel (A-D) lists the stage of the parasite (first line) and the molecular size determination based on immunoblotting (second line). The third line indicates which domains were detected in either the immunoblotting or immune electron microscopy experiments. The immune electron micrographs are excerpted from FIG. 4. The asterisks in B and D indicate that the molecular size is not confirmed by immunoblotting analyses. The question mark (?)in D indicates that the presence of the G domain could not be unequivocally confirmed. All abbreviations are as in FIGS. 3 and 4.

FIG. 6. Immunoblot analyses to assess the antigenicity of MB2 recombinant peptides. A: Schematic representation of the MB2 protein sequence. The three domains, Basic (B), Acidic (A), and GTP-binding (G), are indicated as blocks with the amino acid junctions numbered below. The seven short horizontal lines represent the approximate extent of each of the polypeptides that were expressed as GST-fusion recombinant proteins. B: Immunoblots of GST-MB2 recombinant proteins reacted with anti-GST rabbit serum (Anti-GST); serum of a protected volunteer (#5 volunteer); or serum of a person living in a malaria-endemic area (Endemic serum). Immunoblots were prepared in triplicate, and each lane contains 50-100 ng of purified GST-MB2 recombinant proteins. Recombinant proteins MB2-C, MB2-D and MB2-FA, listed in bold letters, contain amino acid repeats. Approximate molecular weights of the fusion proteins are indicated in kilodaltons (kDa).

FIG. 7. Amino acid sequence alignment showing the size polymorphism in the repeat region of the MB2 gene from different laboratory strains and field isolates. Amino acid positions 211 to 264 make up the repeat domain. Identical amino acids outside the repeat domain are not shown. Field isolates were surveyed from India, Venezuela (Ven), Thailand (Thai) and Papua New Guinea (PNG).

Abbreviations

The abbreviations used herein include: CS, circumsporozoite; CRS, cell-surface retention signal; IEM, immunoelectron microscopy; GST, glutathione S-transferase; NLS, nuclear localization signal; ORF, open reading frame; PV, parasitophosporous vacuole; aa, amino acid(s); UTR, untranslated region; bp, base pair(s); MSP, merozoite surface protein; TRAP, thrombospondin related anonymous protein.

### DETAILED DESCRIPTION OF THE INVENTION

A novel P. falciparum gene, MB2, was identified by screening a sporozoite cDNA library with the serum of a human volunteer protected experimentally by the bites of P. falciparum-infected and irradiated mosquitoes. The single-exon, single-copy MB2 gene is predicted to encode a protein with an Mᵣ of 187,000. The MB2 protein has an amino-terminal basic domain, a central acidic domain, and a carboxyl-terminal domain with similarity to the GTP-binding domain of the prokaryotic translation initiation factor 2. MB2 is expressed in sporozoites, the liver, and blood-stage parasites and gametocytes. The MB2 protein is distributed as an ˜120-kDa moiety on the surface of sporozoites and is imported into the nucleus of blood-stage parasites as an ˜66-kDa species. Proteolytic processing is favored as the mechanism regulating the distinct subcellular localization of the MB2 protein. This differential localization provides multiple opportunities to exploit the MB2 gene product as a vaccine or therapeutic target.

MB2 elicited an immune response detected by serum antibodies in all human volunteers (5/5) that were immunized experimentally and protected by the bites of infected and irradiated mosquitoes. In contrast, no anti-MB2 antibodies were detected in the serum of all irradiated-sporozoite immunized but not protected volunteers (3/3). Anti-MB2 antibodies also were detected in the sera of 83% of the individuals living in a malaria endemic area of Kenya.

Protected volunteers produced antibodies that recognized preferentially a region (b) of the basic domain (B) of MB2 that does not contain repeat sequences of amino acids. In contrast, naturally-infected individuals produced antibodies that recognize preferentially regions of MB2 that contain amino acid repeats. Furthermore, anti-MB2 antibodies against the non-repeat region of the B domain inhibited to a greater extent the invasion in vitro of a hepatoma cell line by sporozoites than did antibodies against regions that contain amino acid repeats.

Sequence analysis of 11 field isolates and four laboratory strains showed that the antigenic region of the B domain of the MB2 gene is conserved absolutely in parasites obtained from different parts of the world.

The molecular and immunogenic properties of MB2 indicate that it is a likely vaccine candidate and drug target for malaria. Recombinant antigens derived from genes whose products are localized to the surface are potential vaccine candidate molecules for eliciting protective immunity. In addition, proteins that localize to the nucleus are potential drug targets. The MB2 gene product has both of these properties.

MB 2 was discovered using a process whereby subtractive hybridization was used in conjunction with specific cDNA libraries to uncover cDNAs and genes that correspond to novel surface antigens on sporozoites, including sporozoites of P. falciparum. The screening procedures combine a process of subtractive hybridization to remove cDNAs corresponding to the immunodominant circumsporozoite protein from a sporozoite library, as well as the screening of the library with the serum of a person protected by the bite of irradiated, P. falciparum-infected Anopheles gambiae.

This molecular approach allows the identification of novel surface proteins on sporozoites. Prior to this approach, the discovery of non-CS genes expressed in sporozoites was fortuitous. The present inventors have overcome the abundance of CS genes by using the subtractive procedures, allowing the identification of non-CS clones.

### I. The MB2 P. falciparum Antigen

The MB2 gene is a single-exon, single-copy gene predicted to encode a protein with an Mᵣ of 187,000. MB2 is expressed in sporozoites, the liver, and blood-stage parasites and gametocytes. The MB2 protein is found on the surface of sporozoites and is imported into the nucleus of blood-stage parasites.

Strategy for discovering novel surface proteins on sporozoites of the human malaria parasite P. falciparum. The present inventors constructed cDNA libraries from PolyA⁺ RNA isolated from sporozoites (spz) of P. falciparum. The approach was to generate from this spz library a sublibrary following subtractive hybridization procedures to remove all cDNAs with sequence homology to the CS gene, according to the method described in Example 1.

To evaluate the efficiency of CS depletion in the subtracted cDNA library, 100 ng of the recovered single-stranded cDNA were amplified with oligonucleotide primers complementary to the cloning vector to generate a heterogeneous mixture of fragments of about 200 to about 1000 bp in size. The products were amplified again with gene-specific primers. Although two marker genes, TRAP (Robson, K. J., et al. (1988) Nature 335, 79-82; Rogers, W. O., et al. (1992) Proc. Natl. Acad. Sci. U.S. A. 89, 9176-9180) and P2 (Fidock, D. A., et al. (1998) Exp. Parasitol. 89, 125-128) were detected, no signal corresponding to the CS gene was amplified, indicating that the subtraction technique was highly efficient (data not shown). The amplification products were subcloned into the UniZap vector and packaged in λ phage, producing a library of 1.45×10⁶ primary phage.

Discovery of MB2, a gene encoding a novel protein present in sporozoites of P. falciparum. The CS-depleted cDNA library was screened with the polyclonal serum isolated from a volunteer who had been protected against P. falciparum by the bites of irradiated, infected mosquitoes, according to the method of Example 2. These procedures allowed the identification of cDNAs corresponding to novel surface proteins. A screening of 1×10⁴ primary phage with the human volunteer serum led to the selection of 18 candidate phage clones. These were re-screened, and 12 phage again were reactive for antibodies.

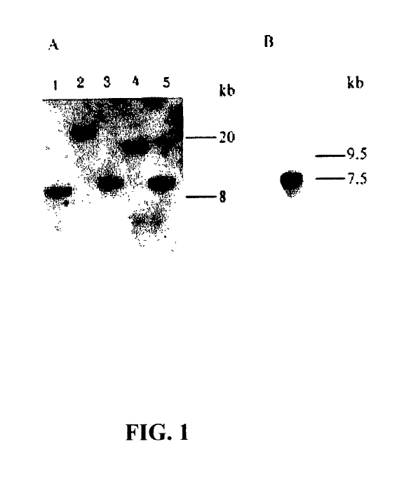

Southern analyses showed that 1 of the 12 secondary clones hybridized specifically to P. falciparum genomic DNA and showed patterns of hybridization consistent with a single-copy gene (FIG. 1A). This 496-bp sporozoite cDNA clone was designated spz-MB2 and was selected for further characterization.

Northern analyses of RNA isolated from blood-stage parasites cultured in vitro and hybridized with probes derived from both the 5′ and 3′ ends of the complete ORF (described below) produced a single positive signal at ˜7.5 kilobases (FIG. 1B).

Sequence analysis of the MB2 cDNA and gene. A comparison of the size of the spz-MB2 cDNA with the mRNA detected in the Northern analyses indicates that it is not a full-length cDNA. Furthermore, primary sequencing of spz-MB2 showed that it lacked a translation termination codon and represented an incomplete ORF.

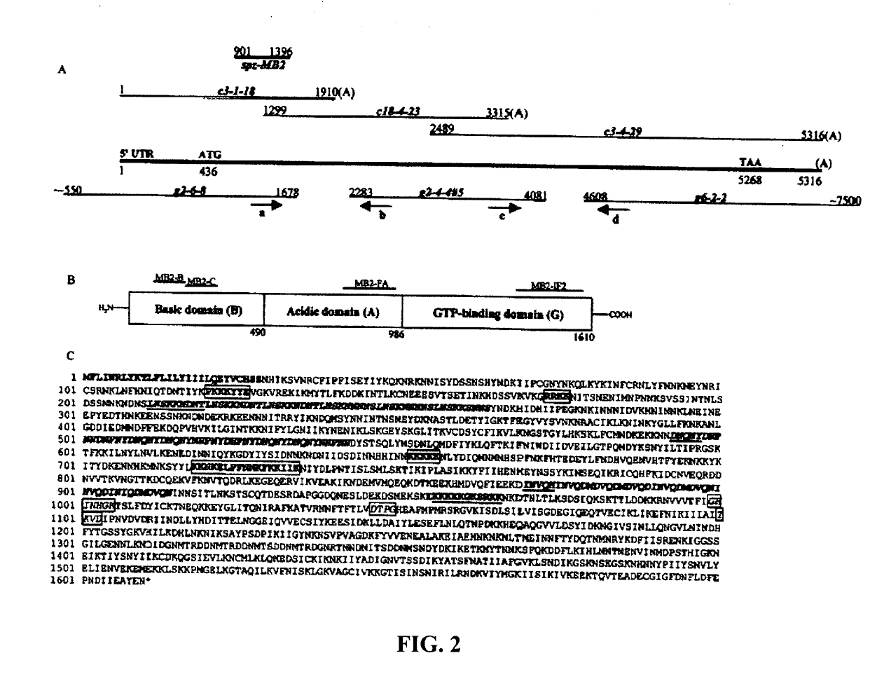

Sequence complementary to MB2 was detected in an asexual blood-stage cDNA library using specific gene amplification primers, and therefore the library was screened with the spz-MB2 cDNA. Two overlapping blood-stage cDNAs, c3-1-18 and c18-4-23, were identified (FIG. 2A).

Nucleotide sequence analysis revealed that the reading frame of spz-MB2 was contained entirely within a contig formed by these two cDNAs. The c3-1-18 clone contained a putative translation initiation codon and a 435-bp 5′ end untranslated region (UTR). The 3′ end termini of the c3-1-18 and c18-4-23 cDNAs each have what appear to be polyadenylation (poly(A)) sequences characteristic of the 3′ end termini of processed mRNAs. However, there were no translation termination codons located to the 5′ end of the poly (A) tracks in either of the cDNAs, and the overlap of c3-1-18 with c18-4-23 revealed that the 17 terminal A nucleotides in c3-1-18 comprise an internal A-rich nucleotide stretch in c18-4-23. Therefore, it was concluded that the oligo(dT) primed the mRNA for cDNA synthesis from within the coding region.

To obtain additional 3′ end sequence of MB2, a Sau3AI genomic library (strain ITO) was screened using as a probe the 400 nucleotides at the 3′ end of c18-4-23. A genomic clone, g2-4-4#5, was identified having overlapping and contiguous sequence with c18-4-23. The sequence of g2-4-4#5 confirmed that the 17-A region at the 3′ end of c18-4-23 is an internal A-rich nucleotide track, supporting the conclusion that these A-rich internal nucleotide tracks were primed by oligo(dT).

To obtain additional 3′ end cDNA sequence, the 600 nucleotides at the 3′ end of g2-4-4#5 were used to screen the blood-stage cDNA library, resulting in the identification of the cDNA clone, c3-4-29. Sequencing of c3-4-29 revealed that it was contiguous with c18-4-23. In addition, there are three stop codons at the 3′ end of c-3-4-29, commencing at nucleotides 5266, 5272, and 5293, and there is a putative poly(A) region near the 3′ end of the last stop codon.

The positions of the stop codons and the authenticity of the poly(A) of the MB2 cDNA were supported by the genomic clone, g6-2-2, identified by screening the genomic library with a probe derived from the 3′ end of c3-4-29. Similarly, the 5′ end UTR and the start codon of MB2 also were verified by the clone g2-6-8, isolated from the genomic library using a probe derived from the 5′ end of c3-1-18.

The overlapping primary sequences of the three blood-stage cDNA clones and the contiguity of their reading frames allowed the assembly of a complete ORF of MB2 that is 4,830 nucleotides in length, of which 77%. of the bases are A-T pairs. No nucleotide polymorphisms were observed among the cDNA and genomic sequences, indicating that there is a single allele of the MB2 gene encoded and expressed in the parasite strains used in our analyses.

Because the nucleotide sequences of the three genomic clones did not overlap, we designed gene amplification primers a, b, c, and d (shown in FIG. 2A and described in Example 3) to assess the contiguity of the MB2 gene in the parasite genome. Amplification products produced by the primer pairs a+b and c+d with parasite genomic DNA as the template gave the predicted product sizes, ˜700 and ˜1000 bp, respectively, indicating that MB2 is organized as a contiguous, single-exon gene in the parasite genome.

Sequence analysis of the MB2 putative translation product. MB2 encodes a putative translation product that is 1610 amino acids (aa) in length with an approximate molecular mass of 187 kDa (FIGS. 2, B and C). The predicted protein is rich in asparagine (15%) and lysine (13%) and is strongly basic with a calculated net charge of +20 at pH 7 and a pI of 8.3.

The primary amino acid sequence can be separated into three distinct, linear domains, the first of which is an amino-terminal basic domain of 490 residues (aa 1-490) with a calculated net charge of +30 and a pI of 9.4. This has been designated the “B” domain. This domain contains a region of six 9-aa imperfect repeats (aa 211-264) with the consensus sequence L, N, S, K, K, N, D/N, N, T/S.

The central acidic domain, designated “A,” encompasses 496 residues (aa 491-986) with a calculated net charge of 26.2 and a pI of 6.1. The boundary between the B and A domains was selected to maximize the basic and acidic properties of the respective domains. The A domain contains two regions of imperfect repeats of 5 amino acids. The first region (aa 493-542) contains 10 repeats with a consensus sequence of D, N, Q/P, N, Y. The second region (aa 870-914) contains nine repeats with a consensus of I/M, N/D, V, Q, D. No similarities to any sequences of known function deposited in the data bases were detected for either the B or A domains.

Finally, a 624-residue carboxyl-terminal domain (aa 987-1610) with sequence similarity to the GTP-binding domain of the prokaryotic translation initiation factor 2 (IF2), as revealed by the BLAST search program (Altschul, S. F., et al. (1997) Nucleic Acids Res. 25, 3389-3402), has been designated “G”. The boundary between the A and G domains was selected based on the start of the regions of similarity of the MB2 protein with known IF2 molecules.

In contrast to its overall hydrophilic nature, the MB2 polypeptide contains at the amino terminus a strongly hydrophobic region (aa 1-25) mapped by a Kyte-Doolittle hydrophobicity plot. The PSORT computer program (Nakai, K., and Kanehisa, M. (1992) Genomics 14, 897-911) predicted an uncleavable signal peptide in the hydrophobic amino-terminal region of MB2. However, the SignalP program (Nielsen, H., et al. (1997) Protein Eng. 10, 1-6) predicted that the signal peptide could be cleaved between a pair of S—S residues at aa 27-28 (FIG. 2C). Currently, there is no experimental data to support one alternative over the other.

The PSORT program also predicted a number of nuclear localization signals (NLS), PKKK (aa 120-123), RRKK (aa 173-176), KKKKK (aa 652-656), and a bipartite NLS, KKNKELPFNN-KFKKIIK (aa 718-734), within the B and A domains. Multiple putative sites for N-glycosylation, N-myristoylation, and phosphorylation were detected by the ScanProsite program (Appel, R. D., Bairoch, A., and Hochstrasser, D. F. (1994) Trends Biochem. Sci. 19, 258-260; data not shown).

There is a polybasic motif, KKKKKGKSRKK (aa 956-966), just before the start of the G domain, that could function as a plasma membrane localization signal as well as a cell-surface retention sequence (CRS) (Hancock, J. F., Paterson, H., and Marshall, C. J. (1990) Cell 63, 133-139). This sequence also could be a putative NLS, although PSORT failed to identify it as such. The similarity of the G domain to the GTP-binding domains of the prokaryotic IF2 proteins includes the conservation of sequence and spacing of three motifs, GX₄GK (aa 999-1005), DX₂K (aa 1046-1049), and NKXD (aa 1100-1104), common to this family of proteins (Dever, T. E., Glynias, M. V., and Merrick, W. C. (1987) Proc. Natl. Acad. Sci. U.S. A. 84, 1814-1818). There is a small variation in the third motif, TKXD, in MB2 as compared with the consensus seen in other G proteins (FIG. 2C).

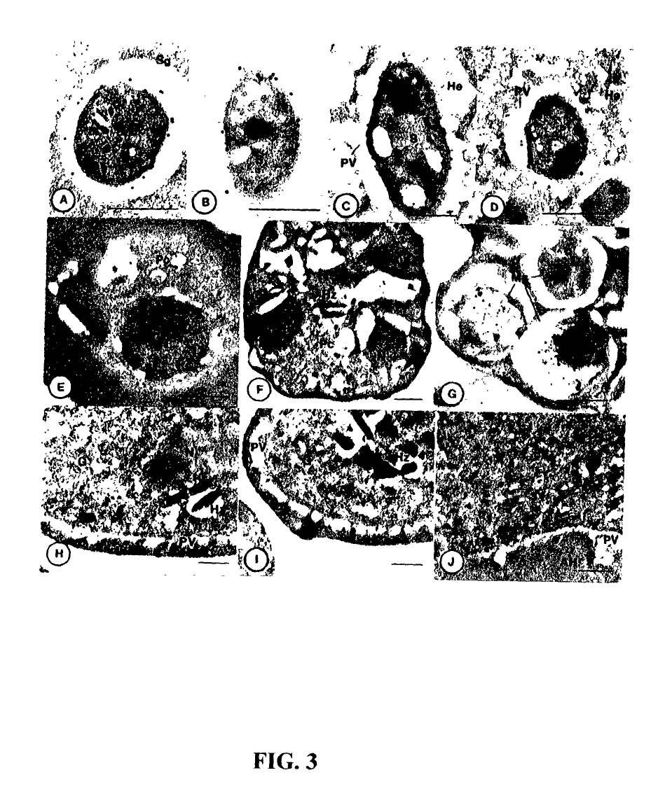

Ultrastructural localization of MB2 protein. Immunoelectron microscopy (IEM) was used to study the subcellular localization of the MB2 antigen. All rabbit antisera prepared against recombinant peptides derived from the B and A domains (FIG. 2A), and reacted with sectioned material containing sporozoites, showed that MB2 protein was localized predominantly to the surface (FIGS. 3, A-D). This was true of sporozoites in salivary glands (FIGS. 3, A and B), as well as those that invaded in vitro cells of the human liver cell line, HepG2-A16 (FIGS. 3, C and D). No antibody reaction was detected with sporozoites using the anti-G domain antibody (data not shown). Preimmune control sera for all reagents were negative (data not shown).

In contrast, the majority of the MB2 protein detected in blood-stage parasites using both antisera against the B domain was localized in the nucleus, with some antibody reactivity detected in the cytoplasm (FIGS. 3, E and F) and data not shown). Rabbit antisera against the A and G domains detected protein only in the cytoplasm of these parasites (FIG. 3G and data not shown). Furthermore, the numbers of gold particles observed in sections of parasites exposed to antibodies against the A and G domains were low when compared with the signal produced by the anti-B domain antisera, suggesting that the majority of MB2 protein present at the blood stages does not contain the A and G domains.

MB2 protein was detected in the cytoplasm, nucleus, and parasitophorous vacuole (PV) space of gametocyte-stage parasites using the anti-B domain antiserum (FIG. 3H). MB2 protein detected by the anti-A domain antiserum was localized only in the PV space (FIG. 3I), indicating that the protein detected in the nucleus and cytoplasm with the anti-B domain antiserum does not contain the A domain. The anti-G domain antiserum produced a high background signal, making it difficult to interpret any specific localization pattern.

Finally, IEM was used to investigate the localization of MB2 protein in the exoerythrocytic stages of the parasite. A section of the liver of an infected Aotus monkey was reacted with the anti-B domain antiserum. Although it is difficult to locate the parasites in these sections, some of the protein was shown to be localized mostly in the cytoplasm with some in the PV space (FIG. 3J). Sections reacted with the anti-A domain antiserum had high backgrounds obscuring any evidence of a specific localization pattern.

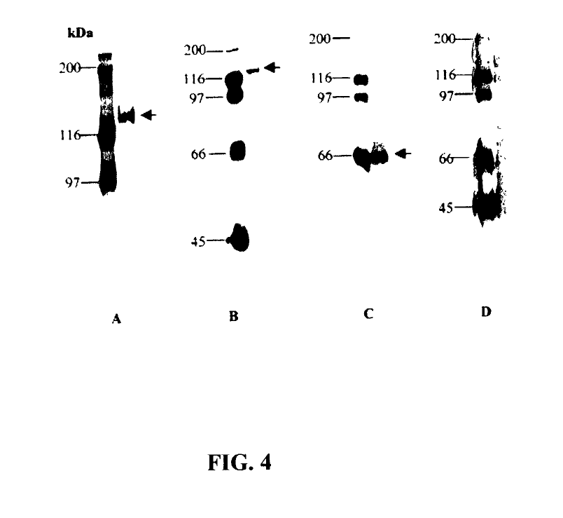

Immunoblot analyses to determine the relative size of the MB2 protein. A series of immunoblotting experiments were performed with parasite protein extracts prepared from the sporozoite and blood stages to determine the relative size of the MB2 protein. The results are shown in FIG. 4. Both anti-B and anti-A domain antisera detected a single polypeptide of ˜120 kDa at the sporozoite stage (FIGS. 4, A and B). No immunoblot analyses were done on sporozoite preparations with anti-G domain antiserum because of the negative results obtained in the IEM analyses.

Immunoblotting using anti-B domain antibody detected a single polypeptide of ˜66 kDa at the blood stages (FIG. 4C). Furthermore, the 66-kDa polypeptide was not detected with either the anti-A or anti-G domain antibodies (FIG. 4D and data not shown). These data are consistent with the IEM study and indicate that the MB2 polypeptide located at the surface of sporozoites consists of only the B and A domains, and the polypeptide translocated into the nucleus of parasites at the blood stages consists primarily of the B domain.

Discussion of MB2 antigen. The Southern analyses shows that MB2 is present in the parasite genome most likely as a single-copy gene. The nucleotide sequence data indicates that it consists of a single exon and is represented most likely in the examined parasite samples by a single allele. The Northern analyses with mRNA obtained from the blood-stage parasites indicates that MB2 is expressed as a single large transcript. It is not known if this size is common to the other developmental stages of the parasite because of the difficulty in obtaining sufficient mRNA for blotting experiments. However, because the gene is single-copy and contains no intron, it is unlikely that multiple transcripts are produced, resulting either from expression of different genes or alternative splicing of a single gene. Thus, it is likely that the single-transcript expression of MB2 is common to other developmental stages.

The overall length of the reconstructed MB2 cDNA is 2.2 kilobases smaller than the RNA species detected in the Northern analyses. This difference likely results from large 5′ end, and perhaps 3′ end, untranslated regions. Although no single genomic nor cDNA clone was identified that spans the entire ORF of MB2, the overlapping primary sequence of the cDNA clones, the contiguity of their reading frames, and the gene amplification analyses of the genomic clones indicate that the complete expressed sequence of the MB2 gene has been identified.

The complete ORF of MB2 predicts a full-length protein of 187 kDa. However, there are many predicted sites for post-translational modification by myristoylation, glycosylation and phosphorylation. Therefore, it is likely that the actual molecular weight of the primary protein structure is increased by processing of individual amino acids. The predicted MB2 protein is rich in asparagine (15%) and lysine (13%), and therefore is strongly basic. Asparagine is the most commonly used (˜12%) amino acid in P. falciparum, followed by lysine and glutamic acid (˜10%) (Hyde, J. E., and Sims, P. F. (1987) Gene 61, 177-187; Weber, J. L. (1987) Gene (Amst.) 52, 103-109). Two other sporozoite surface proteins, CS and the sporozoite-threonine-and-asparagine-rich protein (STARP) (Fidock, D. A., et al. (1994) Mol. Biochem. Parasitol. 64, 219-232), contain 29% and 25% asparagine, respectively. It has been suggested that asparagine-rich motifs in the amino acid sequence might be targets of opsonizing antibodies, promoting parasite phagocytosis by immune cells (Gysin, J., et al. (1993) J. Immunol. Methods 159, 209-219; Barale, J. C., et al. (1997) Mol. Biochem. Parasitol. 87, 169-181; Barale, J. C., et al. (1997) Infect. Immun. 65, 3003-3010). Whether the MB2 protein is a target of opsonizing antibodies is not known, but it is recognized by the immune serum of a human volunteer protected by the irradiated sporozoite vaccine.

Unlike a number of characterized Plasmodium genes that are active only in certain stages, the MB2 gene is expressed in many developmental stages of the parasite life cycle. However, the MB2 gene product has differential localization throughout development. The stage-dependent differential localization of the MB2 protein suggests strongly that it has a multifunctional role during development of the parasites. It is conceivable that it functions as a signal recognition molecule while it is on the surface of the sporozoites. It then may transmit a signal to the nucleus by migrating there during the blood stages. Once inside the nucleus, it may function in the regulation of gene expression, participating in the process of turning off genes that are not required and activating genes that are required for blood stage infection. Examples of genes that are known to be inactivated as the parasite develops to the blood-stage are CS (Suhrbier, A., et al. (1988) Eur. J. Cell Biol. 46, 25-30; Atkinson, C. T., et al. (1989) Am. J. Trop. Med. Hyg. 41, 9-17) and TRAP, and genes that are activated are merozoite surface protein genes, MSPs (Smythe, J. A., et al. (1988) Immunology 85, 5195-5199).

In gametocytes, the protein product is localized in the nucleus, cytoplasm, and the PV space. This differential localization may indicate that the MB2 gene product is in a transitional phase from its functional role in the nucleus to the cell surface or it may have a role in the development of the sexual stages of the parasite. In the exoerythrocytic stage, the MB2 protein detectable by anti-B domain antisera is localized mainly in the cytoplasm, although some can be detected in the PV space. As with the gametocytes, this expression may be a transitional phase in the specific localization as the parasite develops in the liver. The expression of MB2 in this stage is important potentially as a vaccine target since the hepatocyte expresses major histocompatibility complex molecules that can be recognized by T cells. Research in the last 10 years has indicated that the infected hepatocyte can be an important target for immune attack.

Although the MB2 protein is localized on the surface membrane of sporozoites, the primary amino acid sequence contains no apparent transmembrane domain or glycosylphosphatidylinositol anchor signal. However, the amino acid sequence does contain a polybasic motif that was shown to function as plasma membrane localization signal as well as CRS motif (Hancock, supra; Lokeshwar, V. B., Huang, S. S., and Huang, J. S. (1990) J. Biol. Chem. 265, 1665-1675). Many cytokines are retained on the membrane surface of the producer cell in a process mediated by the CRS. Studies have shown that, if the basic amino acids are deleted or mutated to acidic or neutral amino acids, then the membrane localization of the protein is affected (Ostman, A., et al. (1991) Cell Regul. 2, 503-512; Cadwallader, K. A., et al. (1994) Mol. Cell. Biol. 14, 4722-4730). Therefore, the polybasic motif is the most likely domain to be used to localize the MB2 product to the membrane surface of sporozoites.

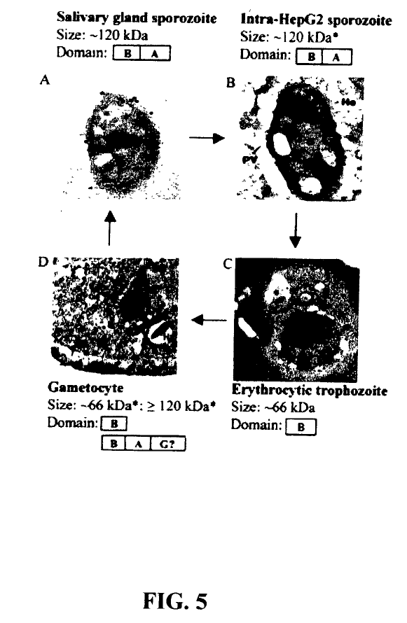

Data suggests that protein processing is the mechanism by which MB2 regulates its differential cellular localization (FIG. 5). The MB2 protein is localized mostly to the surface membrane of sporozoites as an ˜120-kDa species consisting of the B and A domains (FIGS. 3 (A and B) and 5A) It is noted that the predicted molecular mass of the B and A domains combined is ˜116 kDa. This similarity in molecular mass between the actual protein and the predicted domains is remarkably close and does not take into consideration the effects of post-translational changes to specific amino acids or the arbitrary boundaries assigned to the domains.

As the sporozoites invade hepatocytes represented by the human cultured liver cells, the B and A domains of the MB2 protein are still detectable on the membrane surface (FIGS. 3C and D) and 5B). There is no immunoblot data for this stage, but it can be inferred from the IEM study that the size of the protein is most likely ˜120 kDa, similar to the size detected in free sporozoites.

As the parasite develops to the blood stages, the majority of MB2 protein detected inside the parasite nucleus consists only of the B domain and is represented in immunoblots by an ˜66-kDa species (FIGS. 3 (E and F) and 5C). The predicted molecular mass, ˜57 kDa, of the B domain selected by analysis of the amino acid primary structure is consistent with this smaller size polypeptide.

As the parasite differentiates to gametocytes, the MB2 protein is found in the PV space as well as the nucleus and cytoplasm (FIGS. 3 (H and I) and 5D). Based on the different labeling patterns seen with the anti-B and anti-A domain antisera, it is likely that the signal in the cytoplasm and nucleus originates from the ˜66-kDa moiety. The protein in the PV space contains at least the B and A domains, and may contain the G domain. However, as noted in the results, the IEM study using the anti-G domain antibody is inconclusive, and there is no immunoblot data that would provide the size of MB2 for the gametocyte stage.

The MB2 protein detected weakly in the cytoplasm of blood-stage parasites by the anti-A and anti-G domain antisera most likely represents the full-length, newly synthesized MB2 protein that has not been processed proteolytically into the ˜66-kDa polypeptide. The data suggests that the full-length MB2 protein is processed specifically at the sporozoite stage to the ˜120-kDa polypeptide during synthesis and/or cellular trafficking. The ˜120-kDa species contains the polybasic CRS-like motif, allowing it to be preferentially retained on the surface of the sporozoite. The secondary or higher-order structure of the ˜120-kDa protein may conceal the NLS in the B and A domains.

At the blood stage, the full-length MB2 protein is processed specifically into the ˜66-kDa polypeptide as supported by the absence of the ˜120-kDa species. The processing of the MB2 protein into the ˜66-kDa polypeptide would remove the polybasic motif, thus removing the membrane targeting signal, and perhaps this processing exposes the nuclear localization signals allowing the ˜66-kDa polypeptide to translocate to the nucleus. Finally, MB2 protein in the gametocyte stage may be processed into at least two forms, one of which consists of at least the B and A domains and is exported to the PV space. The other form, consisting most likely of only the B domain, is transported to the nucleus.

Another interesting feature of the MB2 protein is the G domain, which has significant sequence similarity to the prokaryotic IF2. The data presented herein indicates that the G domain is not present in the MB2 protein detected on the sporozoite surface, nor is it present in the nucleus at the blood stages. It is conceivable that the cleavage of the MB2 protein requires energy, and this requirement is fulfilled by the G domain since it can bind to GTP. The cleavage process most likely includes removal of the G domain as evidenced by the inability to detect it with specific antiserum in most stages of the parasite. Alternatively, because MB2 can bind potentially to GTP, it is possible that there are conformational differences between the GTP-bound, GDP-bound, and unbound states that can regulate the distinct proteolytic processing of the MB2 protein.

### II. Immunological Characterization P. falciparum Sporozoite Surface Antigen MB2.

The MB2 antigen is a target of antibody response in protected but not unprotected volunteers exposed to the bites of P. falciparum infected and irradiated mosquitoes. The MB2 antigen possesses intriguing immunogenic and molecular properties that indicate that it may be an important immune target for vaccine studies.

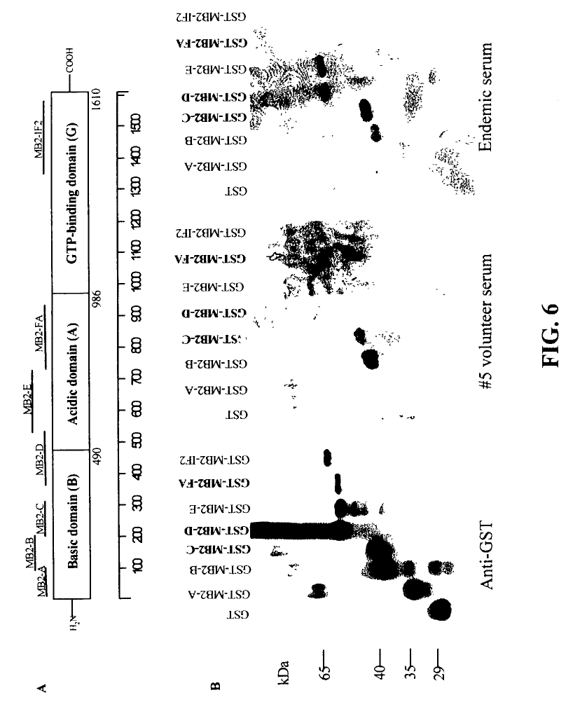

Recombinant protein expression and purification. GST-MB2 recombinant proteins representing various regions of the coding sequence of MB2 were expressed in bacteria, in accordance with the method described in Example 5. The seven MB2 recombinant proteins, MB2-A, MB2-B, MB2-C, MB2-D, MB2-E, MB2-FA and MB2-IF2 are shown schematically in FIG. 6A. (Recombinant proteins MB2-B, MB2-C, MB2-FA and MB2-IF2 are also shown schematically in FIG. 2B.)

Bacteria transformed with plasmids carrying different regions of MB2 required different induction times and culture media for optimal yield, as shown in Table 1. The yield also varied greatly, with some regions being expressed well in bacteria, while others expressed poorly. The inclusion of about 80 to about 85 mM of imidazole in the washing buffer and the overnight wash during isolation, as described in Example 5, was found to be important in improving the purity of the recombinant protein. All of the expressed proteins were soluble and thus purified easily from the cell-free bacterial lysates by one-step nickel column chromatography.

TABLE 1
Optimal conditions to express GST-MB2 recombinant
proteins in E. coil

| | AMINO | | | | | | |
|---|---|---|---|---|---|---|---|
| | ACID | | | | | | |
| | POSITION^{a} | NET | | INDUCTION | | EXPRESSION | |
| NAME | (SIZE) | CHARGE^{b} | MEDIA^{c} | TIME | | LEVEL^{d} | |
| MB-A | 32-101 | +8.94 | SB | 1 | hr | 1-2 | mg/L |
| | (70aa) | | | 10 | min | | |
| MB2-B | 95-206 | +10.18 | LB | 8 | hrs | 3 | mg/L |
| | (112aa) | | | | | | |
| MB2-C | 200-316 | +2.58 | LB | 4 | hrs | 4 | mg/L |
| | (117aa) | | | | | | |
| MB2-F^{e} | 32-316 | +21.91 | SB | 4 | hrs | 2 | mg/L |
| (A-B-C) | (285aa) | | | | | | |
| MB2-D | 355-546 | −0.72 | SB | 1-6 | hrs | 0.1-0.2 | mg/L |
| | (192aa) | | | | | | |
| MB2-E | 538-773 | +8.23 | SB | 1-2 | hrs | 0.1-0.2 | mg/L |
| | (236aa) | | | | | | |
| MB2-FA | 764-945 | −17.51 | SB | 3-6 | hrs | 2 | mg/L |
| | (182aa) | | | | | | |
| MB2-IF2 | 1337-1606 | +5.26 | LB or | 1 | hr | 0.5-1.0 | mg/L |
| | (270aa) | | SB | | | | |

^{a}Position is based on the translation initiation methionine (1).
^{b}Calculated at neutral pH
^{c}LB, Luria Broth; SB, Super Broth. Media were made according to instructions (Sambrook et al., supra)
^{d}Expressed as amount of purified protein per volume of IPTG-induced culture
^{e}Combined length of MB2-A + MB-2B + MB2-C

Immunoblot analyses to determine the antigenic regions of MB2. Purified GST-MB2 recombinant proteins were used for immunoblot analyses to determine the antigenic regions of the molecule. The results of the immunoblot analyses obtained with the serum from a protected volunteer (#5) showed that it contains mostly antibodies against the B domain (FIG. 6B). Minimal or no antibody reactivity was observed against the recombinant fragments of the A or G domains. Moreover, the antigenic property of MB2 is limited to the central region of the B domain, with antibody reacting strongly against the non-repeat-containing MB2-B peptide, and some antibody reactivity observed against the repeat-containing MB2-C peptide.

For comparison, the antigenicity of MB2 also was analyzed using the serum from an individual exposed naturally to P. falciparum (KU162, Table 2). As shown in FIG. 6B, as with the volunteer serum, the endemic serum contains no antibodies against the MB2-IF2 peptide from the G domain. However, in addition to antibodies against the B domain, the endemic serum also contains antibodies against the A domain. Furthermore, within the B domain, in contrast to serum from the protected volunteer #5, the endemic serum contained antibodies that strongly react against the repeat-containing peptide, MB2-C.

TABLE 2
Individual antibody responses to MB2 recombinant
peptides of the B domain

| Serum Source: | MB2-A^{a} | MB2-B^{a} | MB2-C^{a} |
|---|---|---|---|
| Volunteer serum: | | | |
| Volunteer #1 | −^{b} | ++^{b} | −/+^{b} |
| (protected) | | | |
| Volunteer #3 | − | ++ | −/+ |
| (protected) | | | |
| Volunteer #5 | − | ++ | −/+ |
| (protected) | | | |
| Volunteer #7 | − | ++ | −/+ |
| (protected) | | | |
| WRAIR #1 | − | ++ | −/+ |
| (protected) | | | |
| WRAIR #4 (not | − | − | − |
| protected) | | | |
| WRAIR #5 (not | − | − | − |
| protected) | | | |
| WRAIR #6 (not | − | − | − |
| protected) | | | |
| Kenyan serum: | | | |
| Smear negative | | | |
| (adults) | | | |
| ^{c}KU 036 | − | ++ | + |
| KU 071 | − | ++ | +++ |
| KU 081 | − | + | ++ |
| KU 069 | − | + | ++ |
| KU 079 | − | −/+ | ++ |
| KU 083 | − | −/+ | +++ |
| KU 163 | − | ++ | −/+ |
| KU 202 | − | − | + |
| KU 205 | − | −/+ | −/+ |
| Asymptomatic | | | |
| (adults) | | | |
| KU 118 | − | − | − |
| Asymptomatic | | | |
| (children) | | | |
| KU 044 | − | + | − |
| KU 048 | − | − | + |
| KU 076 | − | − | −/+ |
| KU 001 | − | ++ | ++ |
| KU 049 | − | − | − |
| KU 025 | − | − | − |
| Mildly symptomatic | | | |
| (adults) | | | |
| KU 064 | − | − | − |
| KU 070 | − | + | − |
| KU 157 | − | + | +++ |
| KU 158 | − | + | ++ |
| KU 165 | − | − | − |
| KU 172 | − | −/+ | ++ |
| KU 199 | − | + | −/+ |
| KU 203 | − | − | −/+ |
| KU 207 | − | − | + |
| KU 234 | − | + | −/+ |
| Mildly symptomatic | | | |
| (children) | | | |
| KU 162 | − | + | +++ |
| KU 062 | − | − | − |
| KU 161 | − | − | −/+ |
| KU 041 | − | − | −/+ |
| Severely symptomatic | | | |
| (adults) | | | |
| KU 037 | − | ++ | −/+ |
| KU 072 | − | −/+ | −/+ |
| KU 075 | − | + | ++ |
| KU 080 | − | −/+ | + |
| KU 088 | − | − | − |
| KU 145 | − | + | ++ |
| KU 155 | − | − | ++ |
| KU 174 | − | + | + |
| KU 183 | − | −/+ | ++ |
| Severely symptomatic | | | |
| (children) | | | |
| KU 067 | − | − | + |
| KU 084 | − | ++ | + |

^{a}Peptides are described in FIG. 6.
^{b}Plus signs indicate intensity of visible reactive proteins on the immunoblot.
^{c}KU = serum obtained from individuals living in Kenya.

Because the immunoblot analysis showed that the protected volunteer serum recognized principally the two regions in the B domain, and these regions were recognized differently between the volunteer serum and the endemic serum, it was important to determine if this differential antibody recognition correlated with the different immunity observed between protected volunteers and people living in malaria-endemic regions. Thus, the immunoblot analysis was expanded to include serum obtained from 40 individuals living in Kenya with different clinical status from blood-smear negative to severely symptomatic. In addition, serum samples from seven other irradiated sporozoite-immunized volunteers, of which four of seven acquired sterile immunity following being immunized with irradiated sporozoites, were included in the analysis.

Results of the expanded immunoblot analyses are shown in Table 2. A total of 83% (34/41) of naturally exposed individuals have anti-MB2 antibodies. Of these, the analysis showed more individuals had antibodies against MB2-C than MB2-B, 94% vs. 74%, respectively, and the intensity of the antibody reaction against the MB2-C region was almost always stronger than the reaction against the non-repeat, MB2-B region. Minimal or no antibody reaction was detected against the amino-terminal MB2-A region. No obvious association between the anti-MB2 antibody response and clinical immunity in the serum donors was observed.

In comparison, the immunoblot result obtained with sera of irradiated-sporozoite immunized volunteers showed a 100% association between the anti-MB2 antibody response and the immune status of the volunteers (Table 2). All four additional protected volunteers produced anti-MB2 antibodies that are directed preferentially against the non-repeat MB2-B region, a result similar to that obtained with serum of the initial single volunteer. In contrast, none of the three unprotected volunteers produced an antibody response against MB2. Anti-CS antibodies were detected in all volunteer sera by ELISA (data not shown).

Polymorphism assessment of the antigenic region of the MB2 basic domain. Because sequence variation of MB2 could contribute to the different antibody recognition response observed between sera of the volunteers and endemic persons, the antigenic regions in the B domain of MB2 were evaluated for potential amino acid polymorphisms.

The nucleotide sequence of the MB2 gene was determined for field isolates obtained from different malaria-endemic regions of the world. It was expected that samples derived from non-overlapping locales would provide the greatest opportunity to detect sequence variation in MB2. Only the nucleotide sequence encoding the first 317 amino acids of MB2 was examined, as it is the region that contains B-cell epitopes that were recognized differentially in the immunoblot analysis.

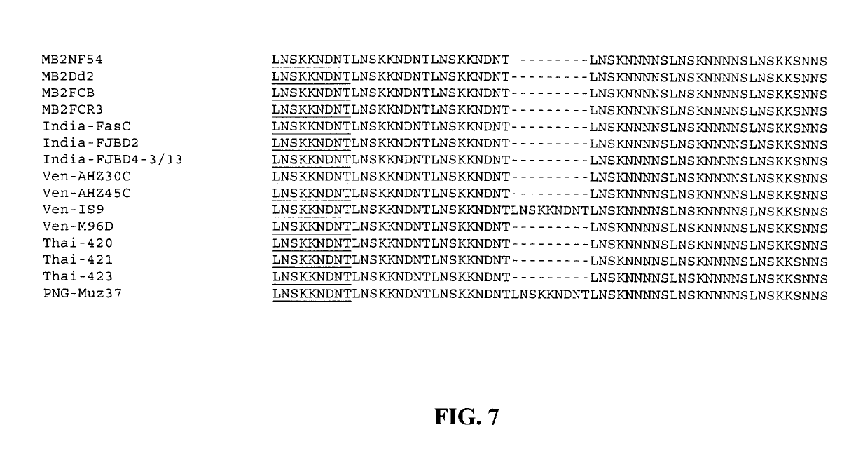

The amino acid sequence alignment of the antigenic region of MB2 from four laboratory strains and eleven field isolates collected from India (3), Venezuela (4), Thailand (3) and Papua New Guinea (1) show that the only variation observed is in the number of repeat units; two out of eleven isolates (Ven-IS9 and PNG Muz37) have 7 versus 6 repeats (FIG. 7). There are no other polymorphisms detected outside the repeat region, indicating that the amino acid sequence of the antigenic region of MB2 is conserved absolutely among the laboratory strains and field isolates examined. The conservation of the antigenic region of the MB2 gene resulted from the conservation of the nucleic acid sequence (data not shown). No silent nucleotide polymorphisms were observed in any of the samples.

In vitro inhibition of sporozoite invasion assay. Because IEM studies indicate that MB2 is located on the membrane surface of sporozoites, the effect of the different antibody recognition against the molecule on the ability of sporozoites to enter hepatocytes was assessed. An in vitro inhibition of sporozoite invasion (ISI) assay was used to study this effect.

Total purified IgG from sera obtained from rabbits immunized against three different regions of MB2 (MB2-B, -C, and -FA) were used in ISI assays. The results show that the IgG against the non-repeat-containing MB2-B peptide has the most inhibitory effect, 57% (Table 3). In contrast, the IgG against the repeat-containing peptide, MB2-C, showed only 18% inhibition. For comparison, the IgG fraction against another repeat-containing but non-antigenic peptide, MB2-FA, showed 33% inhibition.

TABLE 3
Evaluation of in vitro antiparasitic activities
(inhibition of sporozoite invasion) of antibodies against
different regions of MB2

| | SPOROZOITES/ | | |
|---|---|---|---|
| IgG | WELL | MEAN + SD^{a} | % INHIBITION^{b} |
| MB2-B | | | |
| PRE-IMMUNE | 522, 514, 593 | 543 ± 35 | 0 |
| MB2-B | 205, 251, 238 | 231 ± 19 | 57% |
| MB2-C^{c} | | | |
| PRE-IMMUNE | 216, 312, 240 | 256 ± 40 | 0 |
| MB2-C | 252, 177, 198 | 209 ± 31 | 18% |
| MB2-FA^{c} | | | |
| PRE-IMMUNE | 472, 485, 438 | 465 ± 20 | 0 |
| MB2-FA | 263, 354, 306 | 307 ± 37 | 33% |
| Mab NFS1 | 11, 9, 6 | 8 ± 2 | 97% |
| MEM CTRL | 280, 350, 325 | 318 ± 29 | 0 |

^{a}SD, standard deviation
^{b}Inhibition is expressed as % relative to preimmune control culture (0%), and was calculated as follows: 100 × [1 − (mean number of invaded sporozoites in test culture/mean number of invaded sporozoites in control culture)]. Preimmune antibodies were used as negative control.
^{c}These peptides contain amino acid repeats.

Discussion of the immunological characterization of MB2. The novel P. falciparum antigen MB2 is a multi-domain sporozoite surface protein. Immunoblot analyses using serum of a volunteer protected by the irradiated sporozoite vaccine shows that only the basic (B) domain of MB2 is recognized, while the acidic (A) and GTP-binding (G) domains are not. In comparison, serum from a person living in an endemic area contains antibodies against both the B and A domains. It is expected that P. falciparum-exposed individuals should not contain anti-MB2 antibodies directed against the G domain since it is not detected by IEM and immunoblot analyses of sporozoites recovered from mosquito salivary glands as well as from sporozoites that had invaded the hepatoma cell line, HepG2-A16.

The immunoblot analyses revealed that all tested sera from irradiated-sporozoite immunized and protected volunteers contain antibodies against MB2. In contrast, no anti-MB2 antibodies were detected in all tested sera of irradiated-sporozoite immunized but not protected volunteers. Furthermore, antibodies from the serum of protected volunteers recognized preferentially the non-repeat-containing MB2-B peptide, while the repeat-containing MB2-C peptide is recognized preferentially by the serum of persons living in malaria-endemic areas.

In addition, although the A domain also contains two amino acid repeat regions, serum from a protected volunteer showed minimal antibody reactivity against the A domain. In contrast, two regions of the A domain, one of which contains amino acid repeats, were recognized strongly in an endemic serum.

These results indicate that there may be one or more B-cell epitopes encoded in the non-repeat region, peptide MB2-B, that are more relevant to protective immunity than those encoded in the repeat regions. Some authors have hypothesized that B-cell epitopes encoded by the repeat sequence of Plasmodium parasites provide a “smoke-screen” effect to act as a decoy mechanism, shielding other more functionally important epitopes from being detected (Anders, R. F., et al. (1986) Antigenic repeat structures in proteins of Plasmodium falciparum. In: Synthetic peptides as antigens. R. Porter and J. Whelan, eds. Chichester: John Wiley & Sons, pp. 164; Schofield, L. (1990) Bull. World Health Org. 68, Suppl. 66). A similar observation also was reported in the trypomastigote surface antigen-1 (TSA-1) of Trypanosoma cruzi, which contains a repeat region at the carboxyl terminus (Wrightsman, R. A., et al. (1994) J. of Immunology 153, 3148). Animals immunized with either the full-length or the repeat-included carboxyl portion of TSA-1 do not survive a T. cruzi challenge, and the full-length TSA-1 immunized animals only contain anti-TSA-1 antibodies directed against the repeat region. In contrast, a majority of animals immunized with the non-repeat, amino-terminal portion of TSA-1 survive the challenge.

The results of the inhibition of sporozoite invasion assay suggest that antibodies directed against epitopes encoded in the non-repeat region (MB2-B) possess more antiparasitic activities than antibodies against epitopes encoded in the repeat-included region (MB2-C). The in vitro ISI assay showed that anti-MB2-B antibodies are more effective than anti-MB2-C and anti-MB2-FA antibodies at blocking sporozoites from invading the hepatocyte. Although 40% of the parasites are still able to enter hepatoma cells, it is not known how their intra-hepatic development is affected because the sporozoites are not able to complete their liver-stage development in the hepatoma cell used in the ISI assay. The change of the cellular location of MB2 from the surface to the nucleus as the parasite lifecycle progresses from the sporozoite stage to the erythrocytic stage suggests that it may have a function in the development of the parasite. Thus, it is possible that anti-MB2 antibodies, although they fail to block invasion completely, may hinder severely the intra-hepatic development of the sporozoite so that the total effect of anti-MB2 antibodies can completely prevent blood-stage development.

An assay that would provide further support for measuring protective antibodies is the inhibition of liver stage development assay (ILSDA) (Charoenvit, Y., et al. (1997) Infect. & Immun. 65, 3430). The ILSDA requires primary human hepatocytes to support the complete development of the liver stage of P. falciparum sporozoites. Studies utilizing antiMB2 sera in an ILSDA should be illuminating.

Although all five protected volunteers produced anti-MB2 antibodies preferentially against the non-repeat MB2-B region, and the ISI assay showed that anti-MB2-B antibodies are more effective than antibodies against other regions at blocking sporozoite invasion of hepatocytes, it is not known if anti-MB2-B antibodies are protective in vivo and responsible for the sterile immunity acquired by irradiated-sporozoite immunized volunteers. The immunoblot analyses with endemic sera showed no correlation between anti-MB2 antibodies and the clinical status of the infected persons. There are individuals who are blood-smear negative (no evident parasitemia) who have antibodies against the repeat-included MB2-C region, and individuals with parasitemia and disease symptoms who have antibodies against the non-repeat MB2-B region.

It is not known how the clinical status of the infected people was achieved at the time of blood collection. It is possible that their clinical status results from additional factors, such as antimalarial drug use, as well as immune mechanisms. In this case, it would not be meaningful to compare the characteristics of their anti-MB2 antibody response to that of the protected volunteers. Furthermore, clinical immunity was assessed by the presence or absence of parasitemia and clinical symptoms at a single point in time, rather than by prospective analysis of time to infection or incidence of clinical disease.

In addition, it is not known how the B cells of protected volunteers were able to recognize preferentially the non-repeat MB2-B and avoid the repeat-included MB2-C and -D regions that most naturally exposed persons in malaria-endemic areas recognize. It is possible that the antigenic composition of the irradiated sporozoite is different from the normal one. It is known that at the optimal radiation dosage required to induce sterile immunity, the weakened sporozoite is not able to develop completely in the hepatocyte (Sigler, C. I., P. Leland, and M. R. Hollingdale (1984) Am. J. Trop. Med. Hyg. 33, 544). Because the stage-dependent cellular localization of MB2 is accompanied by differential proteolytic processing, it may be that MB2 was not processed properly by the irradiated parasite and is mis-directed onto the surface of infected hepatocytes such that the MB2-B region is now accessible to B cells.

Alternatively, it is observed that protected volunteers received hundreds to more than a thousand bites of infected and irradiated mosquitoes to acquire sterile immunity (Herrington, D., et al. (1991) Am. J. Trop. Med. Hyg. 5, 539-547). In contrast, in malarious countries, most individuals receive on the average less than 200 infective bites per year (Hay, S. I., et al. (2000) Trans R Soc Trop Med Hyg 94, 113) and in the highland area of Kenya, where the endemic serum samples used herein were collected, the number of infective bites is likely to be much lower than this (Khaemba, B., A. Mutani, and M. Bett (1994) East Afr. Med. J. 71, 195). It is possible that, due to an unknown mechanism, the quantitative difference of inoculated sporozoites is responsible for the observed difference in antibody recognition against MB2 between protected volunteers and persons living in endemic countries.

The analysis of the primary structure of amplified MB2 DNA fragments from different isolates of P. falciparum showed that antigenic variation is unlikely to be a factor contributing to the different antibody response against MB2. Except for variation in the number of repeat units, the antigenic region in the B domain of MB2 is absolutely conserved among laboratory strains and field isolates collected from different parts of the world. The amino acid sequence conservation may reflect a functional constraint of the B domain as it is not only exposed on the surface of the sporozoite but also translocated into the nucleus of blood-stage parasites. In the in vitro ISI study, antibodies to this conserved, antigenic region of MB2 inhibit sporozoite invasion of hepatocytes. If these antibodies play a role in protective immunity in vivo, the finding that the antigenic region of MB2 is highly conserved suggests that it is a likely target for immune attack by antibodies as most, if not all, sporozoites would be recognized.

Studies have shown that immunity to malaria also is mediated, at least partly, by cellular immune mechanisms (Hoffman, S. L., et al. (1996) Attacking the Infected Hepatocyte. In: Malaria Vaccine Development. S. L. Hoffman, ed. American Society for Microbiology, Washington, D.C., p. 35). In endemic areas, cytotoxic T lymphocytes (CTLs) from exposed individuals recognize epitopes in a number of pre-erythrocytic antigens of P. falciparum, and indirect evidence indicates that these CTLs may play a role in protective immunity (Aidoo, M., et al. (1995) Lancet 345, 1003; Aidoo, M., et al. (1997) International Immunology 9, 731; Aidoo, M., et al. (2000) Infection and Immunity 68, 227). Because MB2 was shown to be expressed in multiple developmental stages including the hepatic stage that can be recognize by CTLs, it is important to obtain evidence that MB2 also is recognized in P. falciparum-exposed individuals.

The Plasmodium parasite is genetically complex, and based on the recent sequencing projects (Gardner M. J., et al. (1999) Parasitologia 41, 69) may have 5,000-6,000 genes. Its antigenic composition also is expected to be complex. Thus, the challenge in designing an effective recombinant malaria vaccine is to define the immunogenic molecule or molecules that are essential, and the methods to present them properly to the immune system to induce the desired immune responses that protect the volunteers experimentally immunized with the irradiated sporozoite vaccine. The MB2 protein possesses a number of molecular and immunogenic properties that indicate it is a novel immunogen and may represent an important immune subject for vaccine studies.

Nucleotide and Amino Acid Sequences. Sequences identified as SEQ ID Nos 1-9 disclosed in this invention are new. These sequences represent nucleotides and amino acid sequences of P. falciparum antigen. They were prepared according to the methods described in Examples 1-3.

SEQ ID NO 1 is the complete open reading frame (ORF) of MB2, constructed from overlapping sequences of cDNA clones.

SEQ ID NO 2 is cDNA clone c3-1-18.

SEQ ID NO 3 is cDNA clone c18-4-23.

SEQ ID NO 4 is cDNA clone c3-4-29.

SEQ ID NO 5 is cDNA clone Spz-MB2.

SEQ ID NO 6 is genomic clone g2-6-8.

SEQ ID NO 7 is genomic clone g2-4-4#5.

SEQ ID NO 8 is genomic clone g6-2-2.

SEQ ID NO 9 is the amino acid sequence of MB2.

SEQ ID NO 10 is a cDNA clone from isolate NF54.

SEQ ID NO 11 is a cDNA clone from isolate Ven-IS9.

SEQ ID NO 12 is a cDNA clone from isolate PNG-Muz37.

The MB2 cDNA and genomic sequences have been deposited in GenBank, accession numbers AF378132-AF378138 and AF454665-AF454667, inclusive. These sequences are hereby incorporated by reference in their entirety.

In addition to the amino acid sequence of the MB2 polypeptide shown in SEQ ID NO 9, the present invention is also directed to variants and derivatives of this polypeptide.

As used herein, the term “variant” refers to polypeptides in which one or more amino acids have been replaced by different amino acids, such that the resulting variant polypeptide is at least 75% homologous, and preferably at least 85% homologous, to the basic sequence as, for example, shown in SEQ ID NO 9. Homology is defined as the percentage number of amino acids that are identical or constitute conservative substitutions. Conservative substitutions of amino acids are well known in the art. Representative examples are set forth in Table 4.

TABLE 4

| Original Residue | Conservative Substitution(s) |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Val | Ile, Leu |

Variants of polypeptides according to SEQ ID NO 9 may be generated by conventional techniques, including either random or site-directed mutagenesis of DNA encoding SEQ ID NO 9 (for examples, SEQ ID Nos 1-8). The resultant DNA fragments are then cloned into suitable expression hosts such as E. coli using conventional technology and clones that retain the desired activity are detected. The term “variant” also includes naturally occurring allelic variants.

By “derivative” is meant a polypeptide that has been derived from the basic sequence by modification, for example by conjugation or complexing with other chemical moieties or by post-translational modification techniques as would be understood in the art. Such derivatives include amino acid deletions and/or additions to polypeptides according to SEQ ID NO 9 or variants thereof wherein said derivatives retain activity eliciting an immune response. Other derivatives contemplated by the invention include, but are not limited to, modification to side chains, incorporation of unnatural amino acids and/or their derivatives during peptide, polypeptide or protein synthesis and the use of crosslinking agents.

Polypeptides of the inventions may be prepared by any suitable procedure known to those of skill in the art. Recombinant polypeptides of the invention may be produced by culturing a host cell transformed with an expression vector containing nucleic acid encoding a polypeptide, fragment, variant or derivative according to the invention. Recombinant protein may be conveniently prepared by a person skilled in the art using standard protocols as, for example, described in Sambrook, et al., MOLECULAR CLONING. A LABORATORY MANUAL (Cold Spring Harbor Press, 1989), incorporated herein by reference, in particular Sections 16 and 17; Ausubel et al., CURRENT PROTOCOLS IN MOLECULAR BIOLOGY (John Wiley & Sons, Inc. 1994-1998), incorporated herein by reference, in particular Chapters 10 and 16; and Coligan et al., CURRENT PROTOCOLS IN PROTEIN SCIENCE (John Wiley & Sons, Inc. 1995-1997) which is incorporated by reference herein, in particular Chapters 1, 5 and 6. Examples of vectors suitable for expression of recombinant protein include pGEX, pET-9d, pTrxFus or baculovirus (available from Invitrogen). A number of other vectors are available for the production of protein from both full length and partial cDNA and genomic clones, producing both fused or non-fused protein products, depending on the vector used. The resulting proteins are frequently immunologically and functionally similar to the corresponding endogenous proteins.

The obtained polypeptide is purified by methods known in the art or described in Examples. The degree of purification varies depending on the use of the polypeptide. For use in eliciting polyclonal antibodies, the degree of purity may not need to be very high. However, as in some cases impurities may cause adverse reactions, purity of 90-95% is typically preferred and in some instances even required. For the preparation of a pharmaceutical composition, however, the degree of purity must be high, as is known in the art.

Antibodies. The invention also contemplates polyclonal and monoclonal antibodies against the aforementioned polypeptides, fragments, variants and derivatives. Methods of producing polyclonal antibodies are well known to those skilled in the art. Exemplary protocols which may be used are described for example in Coligan et al., CURRENT PROTOCOLS IN IMMUNOLOGY, (John Wiley & Sons, Inc, 1991) which is incorporated herein by reference, and Ausubel et al., (1994-1998, supra), in particular Section III of Chapter 11.

Alternatively, monoclonal antibodies may be produced using the standard method as, for example, described in an article by Kohler and Milstein (1975, Nature 256, 495-497) which is herein incorporated by reference. Anti-P. falciparum polyclonal antibodies recognizing the cloned polypeptide are preferred over a monoclonal antibody (MAb) because they recognize multiple epitopes on the target polypeptide.

This invention further contemplates reagents such as recombinant single-chain or other antibody derivatives, including antibody libraries, prepared using established procedures from mRNAs and/or cDNAs from hybridoma lines expressing antiMB2 antibodies. These reagents also include, but are not limited to, spleens, isolated spleen cells and mRNAs isolated from mice immunized with all or any portion of natural or synthetic MB2 peptides.

According to the method of the current invention, large amounts of recombinant MB2, or derivative, variants or fragments thereof, are produced by scale up processes in commercial plants which enables production of a corresponding large quantity of polyclonal antibodies and/or of immunogen for active immunization. The antibodies to recombinant expressed protein can also be produced according to the invention using the standard method available for production of the antibodies to native protein.

The antibodies of the invention may be used for affinity chromatography in isolating natural or recombinant P. falciparum polypeptides. The antibodies can also be used to screen expression libraries for variant polypeptides of the invention. The antibodies of the invention can also be used to detect P. falciparum infection.

Detection of P. falciparum. The presence or absence of P. falciparum in a patient may determined by isolating a body specimen, such as blood or other bodily fluid, from a patient, mixing an antibody or antibody fragment described above with the biological sample to form a mixture, and detecting a complex of specifically bound antibody or bound fragment in the mixture which indicates the presence of P. falciparum in the sample.

Any suitable technique for determining formation of the complex may be used. For example, immunoassays, such as radioimmunoassays (RIAs), enzyme-linked immunosorbent assays (ELISAs) and immunochromatographic techniques (ICTs) are well known to those of skill in the art. For example, see “CURRENT PROTOCOLS IN IMMUNOLOGY” (1994, supra) which discloses a variety of immunoassays that may be used in accordance with the present invention.

Examples of body specimens are stools and other liquid or solid body output or tissue samples obtained from a subject. Examples of body fluids are blood, serum, saliva, urine, and the like. Methods for the preparation of the body substance and the body fluid are standard in the art and are described, for example in Manual of Clinical Microbiology, Chapter 8, “Collection, Handling and Processing of Specimens”, 4th edition, Eds, Lennette, E. H., Balows, A., Hausler, W. J. and Shadorny, A. J., American Society for Microbiology, (1986)).

Diagnosis and detection methods also comprise contacting the DNA and RNA of body fluid, tissue, specimen and/or environmental sample with DNA and RNA of the invention or fragments thereof and the amplification of this specific interaction via PCR, branched chain nucleic acid technology and other amplification technologies such that the presence of P. falciparum DNA and/or RNA in the bodily fluid, tissue, specimen or environmental sample may be detected.

Agents suitable for immunodiagnostic use are proteins comprising epitopes of P. falciparum that are recognized by intact B and/or T cells. These proteins are produced as described above, purified and used to detect or characterize anti-P. falciparum antibody in the body substances of populations at risk of prior or current P. falciparum infection. In addition, antibodies to such proteins are obtained by immunizing animals, such as cows, rabbits or goats, or birds with the vaccine combined with an adjuvant.

Pharmaceutical compositions. Another aspect of the invention is the use of the MB2 polypeptide, fragment, variant or derivative of the invention (collectively, “immunogenic agents”) as actives in a pharmaceutical composition for protecting patients against infection by P. falciparum. Suitably, the pharmaceutical composition comprises a pharmaceutically acceptable carrier.

A “pharmaceutically-acceptable carrier” is a solid or liquid filler, diluent or encapsulating substance that may be safely used in systemic administration. Depending upon the particular route of administration, a variety of pharmaceutically acceptable carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline, and pyrogen-free water.

The pharmaceutical composition of the invention may be administered by any of the conventional routes of administration, including oral, rectal, parenteral, sublingual, buccal, intravenous, intra-articular, intramuscular, intra-dermal, subcutaneous, inhalational, intraocular, intraperitoneal, intracerebroventricular, transdermal and the like. Also, the pharmaceutical composition of the invention may be in any of several conventional dosage forms, including, but not limited to, tablets, dispersions, suspensions, injections, solutions, capsules, suppositories, aerosols, and transdermal patches.

The above compositions may be used as therapeutic or prophylactic vaccines. Accordingly, the invention extends to the production of vaccines containing as actives one or more of the immunogenic agents of the invention. Any suitable procedure is contemplated for producing such vaccines. Exemplary procedures include, for example, those described in NEW GENERATION VACCINES (1997, Levine et al., Marcel Dekker, Inc. New York, Basel Hong Kong) and U.S. Pat. No. 6,254,869, both of which are incorporated herein by reference.

Thus, the invention describes vaccines able to provide active B cell-immunity and potentially T cell immunity against malaria. Typical intramuscular immunization schedules are as follows. The immunogenic agents, plus equal volume complete pharmaceutically acceptable adjuvants and excipients is used at the beginning of immunization. Immunogenic agents plus equal volume incomplete adjuvant is used at week 2. Immunogenic agents plus equal volume incomplete adjuvant is used at week 4.

DNA or RNA vaccines. DNA or RNA vaccines have been described in Science, 259:1745 (1993), hereby incorporated by reference in its entirety. Briefly, nucleic acid vectors containing DNA or RNA encoding P. falciparum immunogenic agent(s) are injected, preferably intramuscularly, into the host, where the vector is expressed to produced antigen. The antigen elicits immune responses in the form of specific anti-P. falciparum antigen antibody or cell mediated immune events. In this way, the host receives DNA or RNA and provides his or her own humoral immunity and/or cell mediated responses.

Immunotherapy and prophylaxis. A method for immunotherapeutic treatment, retardation, or inhibition of P. falciparum infection comprises administering to a subject in need of such treatment an amount of an anti-P. falciparum polyclonal or monoclonal antibody prepared according to the invention, effective to provide immunity against the invasion of P. falciparum or effective to inhibit the existing P. falciparum infection.

A method of prophylaxis of P. falciparum infection comprises administering to a subject in need of such treatment a vaccine, as described above, comprising the protein or recombinant polypeptide of this invention capable of endogenous development of inhibitory amount of anti-P. falciparum antibodies.

Typical immunization is achieved by inoculation of the animal, bird or human host with the antigen protein combined with adjuvant.

For passive immunotherapy when used to passively immunize P. falciparum infected hosts, the polypeptide is first combined with appropriate adjuvants and used for the immunization of cows or other donor animals to produce antibodies which may be administered to patients with malaria. Monoclonal antibodies produced in animals, in humans “humanized” from animal sources and produced through chimeric techniques and other derivative techniques may be used for passive immunotherapy.

When in a therapeutic composition, the antigen protein is combined with appropriate adjuvants and used for the immunization of patients who are at risk for malaria either at the time of immunization of in the future.

Qualitative and quantitative detection of P. falciparum: formulations and kits. Formulations suitable for the administration of polypeptides and antibodies such as those described herein are known in the art. Typically, other components stimulatory of immune response as well as fillers, coloring, and the like may be added, such as pharmaceutically acceptable excipient, additives and adjuvants.

For qualitative and quantitative determination of the presence of the P. falciparum infection and environmental contamination, a kit for the diagnosis/detection of P. falciparum is used. The kit comprises the polyclonal antibody or antigen of this invention and a means for detecting the completing of the antibody with antigen.

Another such kit comprises DNA/RNA of the invention for use in detecting complementary DNA/RNA of P. falciparum MB2. Another such kit comprises PCR primers for amplification of MB2 sequences and a method of identifying them.

The kit is utilized for the detection of endogenous antibodies/antigens/DNA/RNA produced by a subject that is afflicted with malaria. Even at the early stages where the parasite is commencing invasion of a subject's cells, some amount of the P. falciparum antigen or the specific antibody may be detected in serum. The kit is also utilized for the detection of antigens/DNA/RNA present in the environmental samples, including, for example, testing of mosquitoes for the presence of Plasmodium.

The kit detects either the antigen with the polyclonal antibodies or the presence of the anti-P. falciparum antibody with the antigen. The complexing immunoreaction is detected by staining, radiography, immunoprecipitation or by any other means used in the art and suitable for these purposes.

In addition to the above, the kits may also comprise a control compounds, anti-antibodies, protein A/G, and the like, suitable for conducting the different assays referred to above.

Having generally described the invention, the same will be more readily understood through reference to the following examples, which are provided by way of illustration, and are not intended to be limiting of the present invention.

### Example 1

Library Construction. A CS-depleted sporozoite cDNA library was constructed from a P. falciparum salivary gland sporozoite cDNA library (strain NF54; Fidock, D. A., et al. (2000) Exp. Parasitol. 95, 220-225) using a hydroxyapatite column-based subtractive hybridization technique (Usui, H., et al. (1994) J. Neurosci. 14, 4915-4926). To prepare the target cDNA sense-strands, DNA from the unsubtracted library was linearized with NotI and used as a template to transcribe antisense cRNAs with T7 RNA polymerase (Megascript, Ambion). Template DNA was removed by DNase treatment and the antisense cRNA strands were used to generate cDNA sense strands in a reaction using SuperScript (Life Technologies, Inc.) reverse transcriptase. To prepare the driver cRNA, a CS clone, G89 (McCutchan, T. F., et al. (1984) Science 225, 625-628), was linearized with NotI. Digestion products were used to generate antisense CS cRNA with T7 RNA polymerase (Megascript, Ambion).

The target cDNA sense strands were allowed to reassociate with a 50-fold excess of the driver cRNA antisense strands. The reassociation mix was loaded onto a hydroxyapatite column and nonduplex, single-stranded target cDNA was separated from duplex cDNA/cRNA by elution with a high molarity phosphate buffer. Primers specific for the UniZap λ phage vector (Stratagene) were used to amplify the subtracted cDNA, and the amplification products were subcloned into the phage arms of the UniZap vector and packaged.

### Example 2

Library Screening. Phage were plated and lifted onto nitrocellulose membranes that were soaked in 10 mM isopropylthio-β-D-galactoside and air-dried prior to use. Membranes were incubated in the serum of human volunteer 5 at a 1:100 dilution, and horseradish peroxidase-conjugated anti-human IgG+IgM were used to detect positive antibody reactions by the ECL system (Amersham Pharmacia Biotech). Positive phage were screened a second time to isolate single phage clones. Additional cDNA and genomic clones (strain ITO) were recovered using MB2-derived ³²P-labeled probes and standard library-screening techniques (Sambrook, J., Fritsch, E. F., and Maniatis, T. (1989) Molecular Cloning: a Laboratory Manual, Cold Spring Harbor Laboratory, Cold Spring Harbor, N.Y.).

### Example 3

DNA Sequencing of MB2. The primary nucleotide sequences of all clones were determined by the dideoxynucleotide chain termination method (Sanger, F., Nicklen, S., and Coulson, A. R. (1977) Proc. Natl. Acad. Sci. U.S. A. 74, 5463-5467) using a ³³P nucleotide terminator kit (Amersham Pharmacia Biotech). Specific oligonucleotide primers for sequencing were made by Heligen Laboratories (Huntington Beach, Calif.). Contiguity of clones was verified by gene amplification of genomic DNA using the following primers: a, 5′-GGTGATGACATTGAAGATATGAATG-3′; b, 5′

CAATAGAATAGATATAATCACC; c,
5′-CTGGGTCATCATATGGAAAAGTG-3′; and d,
5′-CAATACACCCTGCAACCTTTCC-3′.

### Example 4

Southern and Northern Analyses. P. falciparum genomic DNA was isolated using a phenol/chloroform-based procedure (Sambrook et al., supra) from blood-stage parasites (strain FCR3) cultured in vitro. The DNA was digested with various restriction endonucleases, and Southern blots were prepared as described (Id.). The probe for Southern blot analyses was prepared by labeling the sporozoite cDNA clone, spz-MB2, with radioactive [³²P]ATP using the Megaprime DNA system (Amersham Pharmacia Biotech). Total RNA was isolated from blood-stage parasites of the same strain cultured in vitro using the TRIZOL® reagent (Life Technologies, Inc.). Total RNA (15-20 μg) was electrophoresed and Northern blots were prepared as described (Id.). Two ³²P-labeled probes consisting of nucleotides 1-580 and 2393-2836 of the coding sequence of MB2 were used separately on filters to which RNA from blood-stage parasites had been transferred.

### Example 5

Recombinant Protein Expression and Purification Fragments of the MB2 open reading frame (ORF) were expressed in bacteria as GST-MB2-6× His fusion proteins in the dual-affinity pAK1-6H expression vector (Stratmann, T., et al. (1997) Protein Expression Purif. 11, 72-78). NcoI and SmaI cloning sites were created for each insert by amplifying NF54 strain genomic DNA. The names, positions, and nucleotide sequences of oligonucleotide primers used to amplify, clone, and express GST-MB2 recombinant proteins are listed in Table 6.

TABLE 6

Names, positions, and sequences of primers used to
clone and express GST-fusion proteins representing
various regions of the MB2 open reading frame.

| | | | |
|---|---|---|---|
| | 5′ primer-Nco I | 3′ primer-Sma I | |
| GST- | (5′ to 3′) + | (5′ to 3′) + | Amino |
| fusion | strand | strand | acid^{a} |

| | | | |
|---|---|---|---|
| MB2-A | ^{b}(98)GATGCCATGGGTG | (305)GATCCCGGGGAGC | 32- |
| | TTAATAGATGTTTTATC | ATATTCTATTATATTCA | 101 |
| MB2-B | (286)GATGCCATGGAAT | (620)GATCCCGGGTTTT | 95- |
| | ATAATAGAATATGCTCA | TATTATTAGAAGAATCA | 206 |
| MB2-C^{C} | (602)GATGCCATGGATT | (953)ATGCATCCCCGGG | 200- |
| | CTTCTAATAATAAAAAT | TCATTTTTTATTTGAAGA | 316 |
| | ATTCTC | | |
| MB2-F^{d} | (98)GATGCCATGGGTGT | (953)ATGCATCCCCGGG | 32- |
| | TAATAGATGTTTTATC | TCTTTTTTATTTGAAGAA | 316 |
| | | TTCTC | |
| MB2-D^{c} | (1066)GATGCCATGGCA | (1640)GATCCCGGGGAT | 355- |
| | TCTACATTAGATGAAACA | GTACTATAATCATTATTT | 546 |
| | | GG | |
| MB2-E | (1616)GATGCCATGGAT | (2321)GATCCCGGGCTT | 538- |
| | CCAAATAATGATTATAGT | GAATTATATTCTTTATTT | 773 |
| | ACA | TCGTG | |
| MB2- | (2294)GTATGCCATGGT | (2837)GATCCCGGGTCA | 764- |
| FA^{c} | CCACGAAAATAAAGAATA | TCGAGCGATTCATTTTGG | 945 |
| | TAATTCAAG | TC | |
| MB2- | (4009)GATGCCATGGAT | (4823)GATCCCGGGTAC | 1337- |
| IF2 | GGTAATAGAACAAATAAT | GCTTCGATTATATCGTTT | 1606 |
| | GAC | GGCTC | |

^{a}Numbers indicate amino acid positions relative to the initiation methionine (1).
^{b}Numbers in parenthesis refer to nucleotide positions relative to the ATG initiation codon; bold letters indicate the restriction endonuclease sites, Nco I and Sma I.
^{c}These peptides includes amino acid repeats.
^{d}This peptide is a combination of MB2-A, -B, and -C.

The amplification products were digested with NcoI and SmaI and ligated into pAK1-6H. The ligation mixture was used to transform Escherichia coli DH10B, and transformants were selected. Bacterial cells were grown at 37° C. in SuperBroth (Life Technologies, Inc.) to an A₆₀₀=0.6 and induced in 1 mM final concentration of isopropylthio-β-D-galactoside for 3 to 4 hours to express recombinant proteins. Purification of recombinant proteins was done using the ProBond resin (Invitrogen) modified by the inclusion of imidazole at 85 mM final concentration in the washing buffer. Eluted fractions were analyzed by SDS-PAGE and immunoblotting for the presence of recombinant proteins using anti-GST antibodies.

### Example 6

Rabbit Immunization. Purified recombinant protein (400 μg) was injected subcutaneously into a rabbit four times at 2-week intervals. Ten days following the last injection, high titer sera were obtained from the rabbit. The sera were depleted of anti-GST antibodies by chromatography on GST-bound nickel columns.

### Example 7

Immunoelectron Microscopy. P. falciparum parasites and parasite-infected cells or tissues were fixed for 30 min at 4° C. with 1% formaldehyde, 0.1% glutaraldehyde in a 0.1 M phosphate buffer, pH 7.4. Fixed samples were washed, dehydrated and embedded in LR White resin (Polysciences, Inc.). Thin sections (70-80 nm) were blocked in a phosphate buffer containing 5% w/v nonfat dry milk and 0.01% v/v Tween 20 (Aikawa, M., and Atkinson, C. T. (1990) Adv. Parasitol. 29, 151-214).

Grids were incubated at 4° C. overnight in solutions containing variable concentrations of rabbit antiserum reactive to domain-specific recombinant proteins diluted in the blocking buffer. Preimmune sera were used as negative controls. After washing, grids were incubated for 1 h in 15 nm gold-conjugated goat anti-rabbit IgG (Amersham Pharmacia Biotech) diluted 1:40 in phosphate buffer containing 1% bovine serum albumin and 0.01% Tween 20. Following the 1 h incubation, grids were rinsed with phosphate buffer containing 1% bovine serum albumin and 0.01% Tween 20 and fixed with glutaraldehyde to stabilize the gold particles. Samples were stained with uranyl acetate and lead citrate and examined by electron microscopy.

### Example 8

Immunoblot Analysis (Rabbit). Protein extracts from parasites were prepared by boiling them in sample buffer for 10 min (Laemmli, U. K. (1970) Nature 227, 680-685). For the sporozoite stage, parasites were isolated from dissected salivary glands of infected mosquitoes. For the asexual blood stages, parasites were obtained from a saponin lysis of infected red blood cells grown in culture (Hyde, J. E., and Read, M. (1993) Methods Mol. Biol. 21, 133-143).

Protein extracts were fractionated on 8% SDS-polyacrylamide gel electrophoresis and transferred onto nitrocellulose membranes. The membranes were incubated in rabbit antiserum diluted 1:500 for 1 hour. Horseradish peroxidase-conjugated anti-rabbit IgG was used to detect positive signals using the ECL kit (Amersham Pharmacia Biotech). Preimmune sera and lysates from uninfected human red blood cells were used as negative controls.

### Example 9

Human Sera. Samples of human serum from individuals exposed naturally to malaria were collected from donors living in Kenya, a malaria-endemic country in East Africa. All donors were exposed previously to P. falciparum infected mosquitoes as evidenced by the presence of anti-CS and anti-TRAP antibodies in their sera.

The clinical status of the serum donors at the time of blood collection was known and was defined in four categories; blood-smear negative and asymptomatic, blood-smear positive and asymptomatic, blood-smear positive and mildly symptomatic, and blood-smear positive and severely symptomatic. Blood-smear negative persons had a lack of evident peripheral blood P. falciparum parasitemia. Blood-smear positive persons had parasites in their red blood cells and were divided into the following categories: severely symptomatic persons had the physical signs of either fever or pallor plus three or more of the following symptoms: antecedent fever, joint pains, headache, chills vomiting, or severe fatigue; mildly symptomatic persons had no fever or pallor, but had two or more of the above symptoms; asymptomatic persons had no fever or pallor, and had none of the above symptoms. Informed consent was obtained from all individuals or their guardians, as described previously (John, C. C., et al. (2000) Infect. Immun. 68, 5198).

Samples of serum from eight volunteers experimentally immunized by the bites of irradiated, infected mosquitoes were obtained from A. Kang, formerly at The Scripps Research Institute (La Jolla, Calif.); from W. O. Rogers at the US Naval Medical Research Center (Rockville, Md.); and from U. Krzych at the Walter Reed Army Institute of Research (Washington D.C.). Five of the eight volunteers were resistant to the challenge of the bites of infected, nonirradiated mosquitoes, and the other three were not (W. Rogers and U. Krzych, personal communication).

### Example 10

Immunoblot Analyses (Human). Purified GST-MB2 recombinant proteins (˜50-100 ng) were resolved by SDS-PAGE in a 12% polyacrylamide gel and the proteins were blotted onto a nitrocellulose membrane. The membrane was reacted with the human serum at a 1:100 dilution in Tris-buffered saline (TBS) plus 0.05% Tween 20 for two hours at room temperature. HRP-conjugated anti-human IgG at 1:80,000 dilution (CalBiochem) was used to detect positive antibody reactions by the ECL system (Amersham). Rabbit anti-GST antibodies were used as a positive control and normal human serum was used as a negative control.

### Example 11

Amplification and Sequence of MB2 Genes from Diverse Parasite Populations. P. falciparum genomic DNA from laboratory-maintained strains and field isolates (kindly provided by Dr. A. Lal, Centers for Disease Control and Prevention, Atlanta, Ga.) was extracted and purified from infected red blood cells. The nucleotides encoding the antigenic region of the B domain of MB2 (amino acids 1-317) were amplified from genomic DNA by the polymerase chain reaction (PCR) using the following primers: 5′-ATGTTTCTAATATGGCGTTTG-3′ and 5′-TCATTTTTATTTGAAGAATT-3′. Conditions for amplification included an initial DNA denaturation of 94° C. for 2 min, followed by 30 cycles at 94° C. for 20 s, 55° C. for 20 s, and 60° C. for 1 min. Genomic DNA (200 ng) from each lab strain was used as template in the reaction. The concentrations of genomic DNAs of field isolates used as template in the amplification reaction were not known due to the limited quantities of material in each of the samples.

The amplification products were cloned directly into a TA cloning vector using the TOPO-PCR cloning kit (Invitrogen) Plasmid DNAs were prepared from bacterial cultures and both strands of the genes were sequenced. Cloning and sequencing were repeated a second time on amplification products obtained independently to ensure reproducibility of the sequence data. Alignment of the sequences was performed by the Clustal method using the Megalign program from the Lasergene computer software. Novel sequences have been deposited in GenBank with accession numbers AF454665-AF454667.

### Example 12

Rabbit Total IgG Purification. Rabbit immunization procedures to obtain polyclonal antibodies against MB2 recombinant proteins were performed as described in Example 6. For IgG purification, the IMMUNOPURE® IgG Protein A Purification Kit was used, and the purification procedure was followed essentially as described by the manufacturer (Pierce, Ill.).

### Example 13

In Vitro Inhibition of Sporozoite Invasion (ISI) Assay. ISI assays were performed to assess the inhibitory effects of antibodies on the invasion of cultured liver cells by sporozoites (Hollingdale, M. R., et al. (1984) Journal of Immunology 132, 909; Charoenvit, Y., et al. (1997) Infect. & Immun. 65, 3430). A human hepatoma cell line, HepG2-A16, (Schwartz, A. L., et al. (1981) J. of Biol. Chem. 256, 8878) was used for the assay.

Approximately 50,000 HepG2-A16 cells were seeded on eight-chamber plastic Lab-Tek slides (Miles Research) in supplemented minimal essential medium as described (Charoenvit, Y., supra). The purified antibodies from Example 12 were added to a final concentration of 100 mg/ml, and HepG2-A16 cells were infected with 25,000 P. falciparum NF54 sporozoites.

Slides were incubated at 37° C. in 5% CO₂ for 3 hours, washed twice with phosphate-buffered saline (PBS), fixed with cold methanol, and rinsed twice again with PBS. Sporozoites that had invaded hepatoma cells were visualized by phase-contrast light microscopy using immunohistochemical staining with a mouse monoclonal antibody (NFS1) against P. falciparum CS protein. Slides were reacted with NFS1 (10 μg/ml) for 30 min at room temperature, followed by incubation with peroxidase-conjugated goat anti-mouse IgG for 30 min at room temperature.

All cultures for ISI assays were done in triplicate with preimmune IgG used as the negative control and the anti-CS monoclonal antibody NFS1 used as the positive control. The NFS1 antibody used for positive control consistently produces a >90% inhibition.

All patents, patent applications, and other publications mentioned in this specification are incorporated herein in their entireties by reference.

While this invention has been described in detail with reference to a certain preferred embodiments, it should be appreciated that the present invention is not limited to those precise embodiments. For example, although one embodiment is described with reference to P. falciparum, homologous MB2 sequences have been detected in other Plasmodium species, including P. yoelli, P. berghei, and P. gallinaceum; it is further anticipated that homologous sequence for MB2 will be detected in P. vivex. Accordingly, this invention encompasses all Plasmodium species containing MB2 homologous sequence.

Rather, in view of the present disclosure which describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

What is claimed is:

1. A purified polypeptide that has an amino acid sequence that is set forth in SEQ ID NO 9.

2. The purified polypeptide of claim 1, wherein the polypeptide is a recombinant polypeptide.

3. A purified polypeptide that is capable of eliciting an immune response against Plasmodium, wherein the polypeptide has an amino acid sequence that is set forth in SEQ ID NO 9.

4. The purified polypeptide of claim 3, wherein the polypeptide comprises at least 50 contiguous amino acid residues.

5. A pharmaceutical composition for eliciting an immune response against Plasmodium, comprising a polypeptide having an amino acid sequence that is set forth in SEQ ID NO 9 and a pharmaceutically acceptable carrier.

6. The pharmaceutical composition of claim 5, wherein the polypeptide comprises at least 50 contiguous amino acid residues.

7. The pharmaceutical composition of claim 5, wherein the polypeptide is encoded by a polynucleotide according to SEQ ID NO 1.

8. A method of eliciting an immune response in a subject against Plasmodium, comprising administering to the subject an isolated polypeptide, wherein the polypeptide has an amino acid sequence that is set forth in SEQ ID NO 9.

9. The method according to claim 8, wherein the polypeptide is encoded by a polynucleotide according to SEQ ID NO 1.

10. The method according to claim 8, wherein the isolated polypeptide comprises at least 50 contiguous amino acid residues.

11. The method according to claim 8, wherein the isolated polypeptide is a recombinant polypeptide.

12. A natural, synthetic or recombinant DNA or RNA vaccine having a nucleotide sequence SEQ ID NO. 1, wherein the vaccine is capable of eliciting development of anti-Plasmodium antibodies.

13. A natural, synthetic or recombinant DNA or RNA vaccine having a nucleotide sequence SEQ ID NO. 2, wherein the vaccine is capable of eliciting development of anti-Plasmodium antibodies.

14. A natural, synthetic or recombinant DNA or RNA vaccine having a nucleotide sequence SEQ ID NO. 3, wherein the vaccine is capable of eliciting development of anti-Plasmodium antibodies.

15. A natural, synthetic or recombinant DNA or RNA vaccine having a nucleotide sequence SEQ ID NO. 4, wherein the vaccine is capable of eliciting development of anti-Plasmodium antibodies.

16. A natural, synthetic or recombinant DNA or RNA vaccine having a nucleotide sequence SEQ ID NO. 5, wherein the vaccine is capable of eliciting development of anti-Plasmodium antibodies.

17. A natural, synthetic or recombinant DNA or RNA vaccine having a nucleotide sequence SEQ ID NO. 6, wherein the vaccine is capable of eliciting development of anti-Plasmodium antibodies.

18. A natural, synthetic or recombinant DNA or RNA vaccine having a nucleotide sequence SEQ ID NO. 7, wherein the vaccine is capable of eliciting development of anti-Plasmodium antibodies.

19. A natural, synthetic or recombinant DNA or RNA vaccine having a nucleotide sequence SEQ ID NO. 8, wherein the vaccine is capable of eliciting development of anti-Plasmodium antibodies.

20. A method for the in vitro diagnosis of malaria, which comprises contacting a body specimen taken from an individual with the purified polypeptide of claim 3 under conditions suitable for binding between the polypeptide and antibodies present in the body specimen; detecting binding between the polypeptide and antibodies; and correlating the binding with the presence of malaria.

21. A kit for in vitro diagnosis of malaria comprising:
the purified polypeptide according to claim 3;
a medium suitable for formation of an antigen-antibody complex; and
reagents for detection of the antigen-antibody complex.

22. A kit for in vitro diagnosis of malaria comprising a set of PCR primers suitable for amplification of MB2.

23. A kit for in vitro diagnosis of malaria comprising:
a purified DNA that has a nucleotide sequence that is set forth in SEQ ID NO 1; and
reagents for detecting complementary DNA or RNA from a body specimen.

24. An antibody that binds specifically to MB2 polypeptide.

25. A method of making an antibody, comprising immunizing a non-human animal with an immunogenic fragment of MB2 polypeptide.

26. A method of making an antibody, comprising providing a hybridoma cell that produces a monoclonal antibody specific for MB2 polypeptide, and culturing the cell under conditions that permit production of the monoclonal antibody.

27. A method of inhibiting Plasmodium infection in a patient, comprising administering to the patient a composition comprising the antibody of claim 24.

28. A method of determining whether a biological sample contains Plasmodium, comprising contacting the sample with the antibody of claim 24 and determining whether the antibody specifically binds to the sample, said binding being an indication that the sample contains Plasmodium.

29. The method of claim 28, wherein the biological sample is a mosquito.

30. The method of claim 28, wherein the biological sample is a human body specimen.

31. A method for purifying MB2 polypeptide from a biological sample containing MB2 polypeptide, comprising
providing an affinity matrix comprising the antibody of claim 24 bound to a solid support;
contacting the biological sample with the affinity matrix, to produce an affinity matrix-MB2 polypeptide complex;
separating the affinity matrix-MB2 polypeptide complex from the remainder of the biological sample; and
releasing MB2 polypeptide from the affinity matrix.

32. A composition comprising purified antigen MB2.

33. A method of eliciting an immune response in an animal, comprising introducing into the animal a composition comprising purified antigen MB2.

34. A method of generating antibodies specific for antigen MB2, comprising introducing into an animal a composition comprising purified antigen MB2.

35. A live bacterial cell vector that (a) infects a human and (b) is stably transformed with, and expresses, a heterologous DNA encoding antigen MB2.

36. A kit for in vitro detection of Plasmodium comprising:
the antibody of claim 24;
a medium suitable for formation of an antigen-antibody complex; and
reagents for detection of the antigen-antibody complex.

37. A kit for in vitro detection of Plasmodium comprising a set of PCR primers suitable for amplification of MB2.

38. A kit for in vitro detection of Plasmodium in a sample comprising:
purified DNA that has a nucleotide sequence that is set forth in SEQ ID NO 1; and
reagents for detecting complementary DNA or RNA from the sample.
